SEQUENCE LISTING (25 sequences)

SEQ ID NO: 1 - DNA, 20 bp, Zea mays
gatgaggctcatcgagcgtc

SEQ ID NO: 2 - DNA, 149 bp, zea mays
tgcatggtacaacaaatagtttgcttagtgatagatgaggctcatcgagcgtcaggaaattatgcatactgcatggttatccgagaggtatgtttcactgtcatgtactacactcatttgttttacctgcaacactaccgtcggatcgt

SEQ ID NO: 3 - protein, 16 aa, zea mays
VIDEAHRASGNYAYCM

SEQ ID NO: 4 - DNA, 150 bp, Artificial Sequence
  Edited FANCM target site
tgcatggtacaacaaatagtttgcttagtgatagatgaggcgtcaggaaattatgcatactgcatggttatccgagaggtatgtttcactgtcatgtactacactcatttgttttacctgcaacactaccgtcggatcgtgcgtgtataa

SEQ ID NO: 5 - protein, 13 aa, Artificial Sequence
  Altered AA sequence at fancm CR4 target
VIDEASGNYAYCM

SEQ ID NO: 6 - DNA, 20 bp, Artificial Sequence
  guide RNA sequence
gtatacataaagctcgtact

SEQ ID NO: 7 - DNA, 23 bp, zea mays
atatacataaagctcgtacttgg

SEQ ID NO: 8 - DNA, 20 bp, Artificial Sequence
  guide RNA sequence
ggtgcagcagtaacctctcc

SEQ ID NO: 9 - DNA, 23 bp, zea mays
agtgcagcagtaacctctccagg

SEQ ID NO: 10 - DNA, 19 bp, Artificial Sequence
  guideRNA
gcgaatttacggcccgaga

SEQ ID NO: 11 - DNA, 23 bp, zea mays
gcgaatttacggcccgagagcgg

SEQ ID NO: 12 - DNA, 20 bp, Artificial Sequence
  GCCTAAATATTTAGTGATCC
gcctaaatatttagtgatcc

SEQ ID NO: 13 - DNA, 23 bp, zea mays
tcctaaatatttagtgatcctgg

SEQ ID NO: 14 - DNA, 20 bp, Artificial Sequence
  guideRNA
gcgtacctggccgccagaac

SEQ ID NO: 15 - DNA, 23 bp, zea mays
gcgtacctggccgccagaaccgg

SEQ ID NO: 16 - DNA, 20 bp, Artificial Sequence
  guideRNA
gttgcaacgtgtcaatcaag

SEQ ID NO: 17 - DNA, 23 bp, zea mays
tttgcaacgtgtcaatcaagcgg

SEQ ID NO: 18 - DNA, 20 bp, Artificial Sequence
  guideRNA
gcaagaatctgctaatgttc

SEQ ID NO: 19 - DNA, 23 bp, zea mays
ccaagaatctgctaatgttccgg

SEQ ID NO: 20 - DNA, 20 bp, Artificial Sequence
  guideRNA
gctggagagtcctacgggta

SEQ ID NO: 21 - DNA, 23 bp, zea mays
gctggagagtcctacgggtacgg

SEQ ID NO: 22 - DNA, 20 bp, Artificial Sequence
  guideRNA
ggattccgcggaaatgggtg

SEQ ID NO: 23 - DNA, 19 bp, Artificial Sequence
  guideRNA
gctacagttgcatttggga

SEQ ID NO: 24 - DNA, 20 bp, Artificial Sequence
  guideRNA
gctcattgtgtaagccagtg

SEQ ID NO: 25 - DNA, 20 bp, Artificial Sequence
  guideRNA
gtggacgtgcgggtagagat

(19) United States
(12) Patent Application Publication
(10) Pub. No.: US 2021/0210163 A1
(43) Pub. Date: Jul. 8, 2021

(54) SYSTEMS AND METHODS FOR IMPROVED BREEDING BY MODULATING RECOMBINATION RATES

(71) Applicant: PIONEER HI-BRED INTERNATIONAL, INC., JOHNSTON, IA (US)

(72) Inventors: ANDREW BAUMGARTEN, JOHNSTON, IA (US); JIN LI, ANKENY, IA (US); GREGORY D MAY, ANKENY, IA (US)

(73) Assignee: PIONEER HI-BRED INTERNATIONAL, INC., JOHNSTON, IA (US)

(21) Appl. No.: 17/057,220

(22) PCT Filed: May 24, 2019
(86) PCT No.: PCT/US19/33907

§ 371 Date: Nov. 20, 2020

Related U.S. Application Data
(60) Provisional application No. 62/676,564, filed on May 25, 2018.
(60) Provisional application No. 62/783,537, filed on Dec. 21, 2018.

(51) Int. Cl. G16B 20/40; C12N 15/82

(52) U.S. Cl. CPC G16B 20/40; C12N 15/8213

(57) ABSTRACT

Systems and methods for improving marker-trait associations and for improving trait introgression precision while reducing trait introgression time are disclosed. Genes responsible for recombination are edited to reduce function, and thus increase recombination rates. Increased recombination rates allow more precise quantification of marker-trait associations, and more precise and faster trait introgression. Methods and compositions useful for selecting an organism with a trait of interest are provided herein. Candidate organisms identified and/or selected by any of the methods described above are also of interest.

### CROSS-REFERENCE TO RELATED APPLICATIONS

This application claims priority to U.S. Provisional Application No. 62/676,564 filed May 25, 2018 and U.S. Provisional Application No. 62/783,537 filed Dec. 21, 2018, each of which is hereby incorporated by reference in their entirety.

### FIELD

The invention relates to breeding methods leveraging enhanced genetic diversity from increasing meiotic recombination rates.

### BACKGROUND

Modern plant and animal breeding methods can use genotyping to make selections based on desired genotype, which reduces breeding cycle time by avoiding the need to grow offspring from a breeding event to maturity to phenotype. Instead, phenotypes can be inferred from known statistical associations with particular genotypes, either through specific phenotypic traits inferred from quantitative trait loci (QTLs), or through a measure of breeding values from whole genome prediction (WGP). Both QTL and WGP methods are based on evidence that the recombination frequency between two chromosome locations is linearly correlated to the length of DNA between the positions. This correlation has been observed in several classical and modern studies where allelic variation in genes in close chromosomal proximity is statistically correlated and recombination events creating new allelic combinations are more limited than in genes with greater chromosomal distances.

Given this relationship between recombination frequency and genomic distance, statistical association between chromosomal and phenotypic variation indicates that the observed chromosomal variation is in proximity to genomic variation contributing to a phenotype. These associations (often called marker-trait associations or MTA) are used by QTL mapping and WGP methods to statistically estimate the chromosomal position of genomic variation contributing to specific phenotypes. MTAs are then used in breeding programs to select for improved phenotypes by surveying and selecting for markers associated to the phenotype.

Breeding programs need to first establish validated MTAs using empirical experiments or existing data before harnessing them for selection. Traditionally, discrete families of individuals are genotyped with genetic markers, phenotyped for a commercially valuable trait, and significant statistical associations determined between genetic markers and phenotypic variation. Conversely, individuals with existing phenotypic data can be genotyped to establish MTAs. Regardless of the method, the quality of the MTA and its effectiveness in selecting for beneficial phenotypes is dependent on the chromosomal distance between the associated marker and the sequence contributing to phenotypic variation. Larger distance between these two loci increases the chance that recombination will decouple the statistical association of allelic variation between the two loci, rendering the association useless for use in additional crosses or generations.

There is a need in the breeding arts for improving statistical associations in MTAs by reducing the impact of recombination events on decoupling the statistical association of allelic variations between associated loci. There is a need in the breeding arts to improve the ability to introgress desirable traits while limiting introgression of undesirable traits using MTAs.

### SUMMARY

Described herein are methods for increasing the association between a genetic marker and an associated genetic trait in an organism. The trait may be any trait of interest. The method may include the step of editing the genome of one or more members of a population of the organism to modulate the activity of one or more genes involved in recombination during meiosis, thereby increasing the meiotic recombination rate or frequency in the population. The methods may also include fertilizing each member of the population to generate a second generation population of offspring. In some aspects, the methods include genotyping each member of the second generation population of offspring using a set of markers associated with a polymorphic genomic region. Members of the second generation population of offspring may be phenotyped for a trait associated with the polymorphic genomic region. In some embodiments, the marker-trait associations are quantified, for example, across the second generation population of offspring to determine a change in the association between the genetic marker and the associated marker-trait associations. The marker-trait associations may be increased, decreased or remain unchanged. In some embodiments, the marker-trait associations may have increased linkage, increased statistical association or correlation, or combinations thereof. In some embodiments, the organism is a plant, mammal, insect, microorganism, or any other organism of interest. In some embodiments, the methods are performed on one or more maize plants. In some embodiments, the fertilizing step is by self-pollinating.

Disclosed herein are methods of selecting an organism with a trait of interest. The trait may be any trait of interest. In some aspects, the organism is without limitation a plant, animal, insect, microorganism, or other microorganism of interest. In some embodiments, the method includes providing a data set that includes genotypic data, phenotypic data, or combinations thereof. The data may be obtained from (i) a population of organisms where one or more organisms in the population comprise one or more introduced genetic modifications that increase meiotic recombination in one or more organisms as compared to a control organism that does not comprise the one or more introduced genetic modifications and/or (ii) a population of organisms derived from a parental population. The parent population includes one or more parental organisms that comprise one or more introduced genetic modifications that increase meiotic recombination in one or more parental organisms as compared to a control organism that does not contain the one or more genetic modifications. The organisms may be from a backcross population or a segregating population.

In some aspects, the method includes identifying or generating one or more marker-trait associations in the data set that correlate with the trait of interest in the population of organisms.

The population of organisms has one or more phenotypic markers, genotypic markers, or combinations thereof. In some aspects, the population of organisms exhibits one or more phenotypic or genotypic markers as a result of one or more introduced genetic modifications that increase meiotic recombination as compared to a control organism that does not contain said introduced genetic modifications. In some embodiments, the marker-trait associations may be newly conferred.

In some embodiments, the marker-trait associations may be increased, decreased or remain unchanged. In some embodiments, the marker-trait associations may have increased linkage, increased statistical association or correlation, or combinations thereof as compared to the corresponding marker-trait association in a control.

In some embodiments, the method includes screening, selecting, or identifying a candidate organism, a population of candidate organisms or genotypic data and/or phenotypic data thereof for the presence or absence of the one or more marker-trait associations that correlate with the trait of interest. In some embodiments, the candidate organism or the population of candidate organisms (i) do not contain the introduced genetic modifications and/or (ii) are not obtained from the organism or the population of organisms that contain or contained the one or more introduced genetic modifications. In some embodiments, meiotic recombination is increased across the genome or across a substantial portion of the genome of the organism or population organisms as compared to a control that does not contain the introduced genetic modifications. In some aspects, the increased meiotic recombination is an increase in meiotic recombination frequency or meiotic cross-over events across the whole genome or a portion of the genome as compared to a control.

The candidate organism or the population of candidate organisms may be selected based the presence or absence of the one or more marker-trait associations that correlate with the trait of interest. In some embodiments, the marker-trait association is a known or predicted negative association between a marker and the trait of interest. In some aspects, the method includes selecting the candidate organism or the population of candidate organisms based on the absence of a negative association.

In some embodiments, the marker-trait association is a known or predicted positive association between a marker and the trait of interest. In some aspects, the method includes selecting the candidate organism or the population of candidate organisms based on the presence of a positive association.

In some embodiments, the genotypic data is nucleotide variation data. The variation data may include but is not limited to a single nucleotide polymorphism (SNP), haplotype, simple sequence repeat (SSR), microRNA, siRNA, quantitative trait loci (QTL), transgene, deletion, mRNA, methylation pattern, or gene expression pattern, or any combinations thereof.

In some embodiments, the nucleotide variation data may include but is not limited to genotypic data from one or more of the following: Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof. In some embodiments, the data set includes but is not limited to genome wide nucleotide variation-phenotype associations.

In some embodiments, when the organism is a plant, the phenotypic data includes but is not limited to data on yield, such as yield gain, grain yield, silage yield, root lodging resistance, stalk lodging resistance, brittle snap resistance, ear height, ear length, kernel rows, kernels per row, kernel size, kernel number, grain moisture, plant height, density tolerance, pod number, number of seeds per pod, maturity, time to flower, heat units to flower, days to flower, disease resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, or combinations thereof.

In some embodiments, the organism, including without limitation a plant, animal, insect, microorganism, or other microorganism of interest, is modified to have increased meiotic recombination by genetically introducing one or more nucleotide substitutions, additions and/or deletions in the organism's genome to increase the activity of one or more genes that function to promote meiotic recombination. In some aspects, the method includes genetically introducing one or more polynucleotides in the organism's genome to increase the expression level or activity of one or more genes that function to promote meiotic recombination. In some aspects, the genes that that function to promote meiotic recombination include without limitation HEI10, MSH4/MSH5 MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, homologs thereof, orthologs thereof, or combinations thereof.

In some embodiments, the organism, including without limitation a plant, animal, insect, microorganism, or other microorganism of interest, is modified to have increased meiotic recombination by genetically introducing one or more nucleotide substitutions, additions and/or deletions in the organism's genome to reduce the activity of one or more genes that function to inhibit meiotic recombination. In some aspects, the one or more genes that function to inhibit recombination include but are not limited to FANCM, MHF1, MHF2, FIDGETIN-LIKE1, RECQ4, TOPOISOMERASE3a, RMI1, RMI2, RTEL1, homologs thereof, orthologs thereof, or combinations thereof.

The one or more nucleotide substitutions, additions and/or deletions may be introduced using any suitable technology or approach. In some aspects, the one or more nucleotide substitutions, additions and/or deletions is introduced using genome-editing technology. In some examples, the genome-editing technology includes an endonuclease including but not limited to Cas/CRISPR, meganuclease, zinc finger nucleases (ZFNs), or transcription activator-like effector nucleases (TALENs), or combinations thereof. In some aspects, the one or more nucleotide substitutions, additions and/or deletions is introduced using irradiation, chemical mutagenesis, or transposons. In some embodiments, the one or more organisms, for example, such as plants, microorganisms, insects, or animals, is modified to have increased meiotic recombination by using RNA technology to suppress the activity of one or more genes that function to inhibit meiotic recombination. Any suitable RNA suppression technology may be used including but not limited to RNAi, microRNA, shRNA, or combinations thereof. In some aspects, the method includes growing the selected candidate organism or the population of candidate organisms.

Also provided herein is a method of selecting an organism with a trait of interest that includes the step of selecting a candidate organism based the presence or absence of the one or more marker-trait associations that correlate with the trait of interest. In some aspects, the organism is a plant, animal, insect, or microorganism, or other organism of interest. In some embodiments, the methods are performed on one or more maize plants. The trait may be any trait of interest.

The marker-trait association may be from a data set comprising genotypic and/or phenotypic data obtained from (i) a population of organisms where one or more organisms in the population comprise one or more introduced genetic modifications that increase meiotic recombination in one or more organisms as compared to a control organism that does not comprise the one or more introduced genetic modifications and/or (ii) a population of organisms derived from a parental population where one or more of the parental organisms contain or contained one or more introduced genetic modifications that increases meiotic recombination as compared to a control organism that does not contain the genetic modification. In some aspects, when the organism is a plant, the one or more plants in the population includes a doubled haploid plant, inbred, hybrid plant, offspring thereof, or a combination thereof. The plant may be from a backcross population or segregating population.

The population of organisms has one or more phenotypic markers, genotypic markers, or combinations thereof. In some aspects, the population of organisms exhibits one or more phenotypic or genotypic markers as a result of one or more introduced genetic modifications that increase meiotic recombination as compared to a control organism that does not contain said introduced genetic modifications. In some embodiments, the marker-trait associations may be newly conferred. In some embodiments, the marker-trait associations may be identified, generated, or updated, or combinations thereof.

In some embodiments, the marker-trait associations may be increased, decreased or remain unchanged. In some embodiments, the marker-trait associations may have increased linkage, increased statistical association or correlation, or combinations thereof as compared to the corresponding marker-trait association in a control.

In some embodiments, meiotic recombination is increased across the genome or across a substantial portion of the genome of the organism or population organisms as compared to a control that does not contain the introduced genetic modifications. In some aspects, the increased meiotic recombination may be an increase in meiotic recombination frequency or meiotic cross-over events across the whole genome or a portion of the genome as compared to a control.

The candidate organism or the population of candidate organisms may be selected based the presence or absence of the one or more marker-trait associations that correlate with the trait of interest. In some embodiments, the marker-trait association is a known or predicted negative association between a marker and the trait of interest. In some aspects, the method includes selecting the candidate organism or the population of candidate organisms based on the absence of a negative association.

In some embodiments, the marker-trait association is a known or predicted positive association between a marker and the trait of interest. In some aspects, the method includes selecting the candidate organism or the population of candidate organisms based on the presence of a positive association.

In some embodiments, the genotypic data is nucleotide variation data. The variation data may include but is not limited to a single nucleotide polymorphism (SNP), haplotype, simple sequence repeat (SSR), microRNA, siRNA, quantitative trait loci (QTL), transgene, deletion, mRNA, methylation pattern, or gene expression pattern, or any combinations thereof.

In some embodiments, the nucleotide variation data may include but is not limited to genotypic data from one or more of the following: Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof. In some embodiments, the data set includes but is not limited to genome wide nucleotide variation-phenotype associations. The trait may be any trait of interest.

In some embodiments, when the organism is a plant, the phenotypic data includes but is not limited to data on yield, such as yield gain, grain yield, silage yield, root lodging resistance, stalk lodging resistance, brittle snap resistance, ear height, ear length, kernel rows, kernels per row, kernel size, kernel number, grain moisture, plant height, density tolerance, pod number, number of seeds per pod, maturity, time to flower, heat units to flower, days to flower, disease resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, or combinations thereof.

In some embodiments, the organism, including without limitation a plant, animal, insect, microorganism, or other microorganism of interest, is modified to have increased meiotic recombination by genetically introducing one or more nucleotide substitutions, additions and/or deletions in the organism's genome to increase the activity of one or more genes that function to promote meiotic recombination. In some aspects, the method includes introducing one or more polynucleotides in the organism's genome to increase the expression level or activity of one or more genes that function to promote meiotic recombination. In some aspects, the genes that that function to promote meiotic recombination include without limitation HEI10, MSH4/MSH5 MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, homologs thereof, orthologs thereof, or combinations thereof.

In some embodiments, the organism, including without limitation a plant or animal, is modified to have increased meiotic recombination by genetically introducing one or more nucleotide substitutions, additions and/or deletions in the organism's genome to reduce the activity of one or more genes that function to inhibit meiotic recombination. In some aspects, the one or more genes that function to inhibit recombination include but are not limited to FANCM, MHF1, MHF2, FIDGETIN-LIKE1, RECQ4, TOPOISOMERASE3a, RMI1, RMI2, RTEL1, homologs thereof, orthologs thereof, or combinations thereof.

The one or more nucleotide substitutions, additions and/or deletions may be introduced using any suitable technology or approach. In some aspects, the one or more nucleotide substitutions, additions and/or deletions is introduced using genome-editing technology. In some examples, the genome-editing technology includes an endonuclease including but not limited to Cas/CRISPR, meganuclease, zinc finger nucleases (ZFNs), or transcription activator-like effector nucleases (TALENs), or combinations thereof. In some aspects, the one or more nucleotide substitutions, additions and/or deletions is introduced using irradiation, chemical mutagenesis, or transposons. In some embodiments, the one or more organisms, for example, such as plants, microorganisms, insects, or animals, is modified to have increased meiotic recombination by using RNA technology to suppress the activity of one or more genes that function to inhibit meiotic recombination. Any suitable RNA suppression technology may be used including but not limited to RNAi, microRNA, shRNA, or combinations thereof.

In some aspects, the method includes growing the selected candidate organism or the population of candidate organisms.

Provided herein are methods of selecting a plant with a trait of interest. In some aspects, the plant is a dicot or a monocot plant. In some embodiments, the method includes providing a data set that includes genotypic data, phenotypic data, or combinations thereof.

The data may be obtained from (i) a population of plants where one or more plants in the population comprise one or more introduced genetic modifications that increase meiotic recombination in one or more plants as compared to a control plant that does not comprise the one or more introduced genetic modifications and/or (ii) a population of plants derived from a parental population wherein one or more of the parental plants contains one or more introduced genetic modifications that increases meiotic recombination in one or more of the parental plants as compared to a control plant that does not contain the genetic modification. In some aspects, one or more plants in the population include a doubled haploid plant, inbred, hybrid plant, offspring thereof, or a combination thereof. The plant may be from a backcross population or segregating population. The population of plants has one or more phenotypic markers, genotypic markers, or combinations thereof.

In some aspects, the method includes identifying or generating one or more marker-trait associations in the data set that correlate with the trait of interest in the population of plants. In some aspects, the population of organisms exhibits one or more phenotypic or genotypic markers as a result of one or more introduced genetic modifications that increase meiotic recombination as compared to a control plant that does not contain introduced genetic modifications. In some embodiments, the marker-trait associations may be newly conferred.

In some embodiments, the marker-trait associations may be increased, decreased or remain unchanged. In some embodiments, the marker-trait associations may have increased linkage, increased statistical association or correlation, or combinations thereof as compared to the corresponding marker-trait association in a control.

In some embodiments, the method includes screening, selecting, or identifying a candidate plant, a population of candidate plants or genotypic data and/or phenotypic data thereof for the presence or absence of the one or more marker-trait associations that correlate with the trait of interest. In some embodiments, the candidate plant or the population of candidate plants (i) do not contain the introduced genetic modifications and/or (ii) are not obtained from the population of plants that contain or contained the one or more introduced genetic modifications. In some embodiments, meiotic recombination is increased across the genome or across a substantial portion of the genome of the plant or the population plants as compared to a control that does not contain the introduced genetic modifications. In some aspects, the increased meiotic recombination may be an increase in meiotic recombination frequency or meiotic cross-over events across whole genome or a portion of the genome as compared to a control.

The candidate plant or the population of candidate plants may be selected based the presence or absence of the one or more marker-trait associations that correlate with the trait of interest. In some embodiments, the marker-trait association is a known or predicted negative association between a marker and the trait of interest. In some aspects, the method includes selecting the candidate plant or the population of candidate plants based on the absence of a negative association.

In some embodiments, the marker-trait association is a known or predicted positive association between a marker and the trait of interest. In some aspects, the method includes selecting the candidate plant or the population of candidate plants based on the presence of a positive association.

In some embodiments, the genotypic data is nucleotide variation data. The variation data may include but is not limited to a single nucleotide polymorphism (SNP), haplotype, simple sequence repeat (SSR), microRNA, siRNA, quantitative trait loci (QTL), transgene, deletion, mRNA, methylation pattern, or gene expression pattern, or any combinations thereof.

In some embodiments, the nucleotide variation data may include but is not limited to genotypic data from one or more of the following: Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof. In some embodiments, the data set includes but is not limited to genome wide nucleotide variation-phenotype associations.

The trait may be any trait of interest. In some embodiments, the trait of interest is a set of observable characteristics based on genetic, environmental, or genetic by environmental interactions. In some aspects, the trait of interest includes but is not limited to color, yield, gene expression, chromatin expression, ear height, ear length, kernel rows, kernels per row, disease resistance, stress resistance, herbicide tolerance, or flowering time.

In some embodiments, when the organism is a plant, the phenotypic data includes but is not limited to data on yield, such as yield gain, grain yield, silage yield, root lodging resistance, stalk lodging resistance, brittle snap resistance, ear height, ear length, kernel rows, kernels per row, kernel size, kernel number, grain moisture, plant height, density tolerance, pod number, number of seeds per pod, maturity, time to flower, heat units to flower, days to flower, disease resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, or combinations thereof.

In some embodiments, the plant is modified to have increased meiotic recombination by genetically introducing one or more nucleotide substitutions, additions and/or deletions in the plant's genome to increase the activity of one or more genes that function to promote meiotic recombination. In some aspects, the method includes introducing one or more polynucleotides in the plant's genome to increase the expression level or activity of one or more genes that function to promote meiotic recombination. In some aspects, the genes that that function to promote meiotic recombination include without limitation HEI10, MSH4/MSH5, Mlh1/Mlh3, MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, homologs thereof, and orthologs thereof, or combinations thereof.

In some embodiments, the plant is modified to have increased meiotic recombination by genetically introducing one or more nucleotide substitutions, additions and/or deletions in the plant's genome to reduce the activity of one or more genes that function to inhibit meiotic recombination. In some aspects, the one or more genes that function to inhibit recombination include but are not limited to FANCM, MHF1, MHF2, FIDGETIN-LIKE1, RECQ4, TOPOISOMERASE3a, RMI1, RMI2, RTEL1, homologs thereof, orthologs thereof, or combinations thereof.

The one or more nucleotide substitutions, additions and/or deletions may be introduced using any suitable technology or approach. In some aspects, the one or more nucleotide substitutions, additions and/or deletions is introduced using genome-editing technology. In some examples, the genome-editing technology includes an endonuclease including but not limited to Cas/CRISPR, meganuclease, zinc finger nucleases (ZFNs), or transcription activator-like effector nucleases (TALENs), or combinations thereof. In some aspects, the one or more nucleotide substitutions, additions and/or deletions is introduced using irradiation, chemical mutagenesis, or transposons. In some embodiments, the one or more plants is modified to have increased meiotic recombination by using RNA technology to suppress the activity of one or more genes that function to inhibit meiotic recombination. Any suitable RNA suppression technology may be used including but not limited to RNAi, microRNA, shRNA, or combinations thereof.

Any plant may be used in the methods provided herein, including but not limited to a soybean, maize, sorghum, cotton, canola, sunflower, rice, wheat, sugarcane, alfalfa tobacco, barley, cassava, peanuts, millet, oil palm, potatoes, rye, or sugar beet plant. In some embodiments, the methods include a plant that is a soybean, maize, sorghum, cotton, canola, sunflower, rice, wheat, sugarcane, alfalfa tobacco, barley, cassava, peanuts, millet, oil palm, potatoes, rye, or sugar beet plant. Accordingly, any population of plants may used with the methods provided herein, including but not limited to a population of soybean, maize, sorghum, cotton, canola, sunflower, rice, wheat, sugarcane, alfalfa tobacco, barley, cassava, peanuts, millet, oil palm, potatoes, rye, or sugar beet plants. In some embodiments, the genotypic data and/or phenotypic data is obtained from a population of soybean, maize, sorghum, cotton, canola, sunflower, rice, wheat, sugarcane, alfalfa tobacco, barley, cassava, peanuts, millet, oil palm, potatoes, rye, or sugar beet plants. In some embodiments, the method includes screening, selecting, or identifying a population of candidate plants, or genotypic data and/or phenotypic data thereof from a population of candidate soybean, maize, sorghum, cotton, canola, sunflower, rice, wheat, sugarcane, alfalfa tobacco, barley, cassava, peanuts, millet, oil palm, potatoes, rye, or sugar beet plants. In some embodiments, the population of plants includes plants from a doubled haploid, inbred plants, hybrid plants, or combinations thereof. In some embodiments, the population of candidate plants includes seeds produced from a cross of two inbred parental plants.

In some aspects, the method includes growing the selected candidate plant or the population of candidate plants.

Provided herein are methods of selecting an organism with a trait of interest or selecting an organism with a desired genotype. In some embodiments, the organism is a plant, mammal, insect, microorganism, or any other organism of interest. In some embodiments, the methods are performed on one or more maize plants. The trait may be any trait of interest.

In some aspects, the method includes providing a data set comprising genotypic and/or phenotypic data obtained from a population of organisms. The one or more organisms in the population (i) exhibit a modulated recombination pattern as compared to a control organism due to a recombination modulation factor and/or (ii) are progeny of one or more parental organisms that exhibit modulated meiotic recombination due to a recombination modulation factor, as compared to a control organism, and where the population of organisms comprises one or more phenotypic or genotypic markers or combinations thereof. One or more marker-trait associations in the data set that correlate with the trait of interest or the desired genotype in the population of organisms may be identified or generated.

In some aspects, the population of organisms exhibits one or more phenotypic or genotypic markers as a result of one or more introduced genetic modifications that increase meiotic recombination as compared to a control organism that does not contain said introduced genetic modifications. In some embodiments, the marker-trait associations may be newly conferred.

In some embodiments, the marker-trait associations may be increased, decreased or remain unchanged. In some embodiments, the marker-trait associations may have increased linkage, increased statistical association or correlation, or combinations thereof as compared to the corresponding marker-trait association in a control.

The method also includes screening a candidate organism or a population of candidate organisms for the presence or absence of the one or more marker-trait associations that correlate with the trait of interest, where the candidate organism or a population of candidate organisms (i) do not comprise the modulated recombination pattern due to the modulation factor and/or (ii) are not the progeny of parental organisms that exhibited modulated meiotic recombination due to a recombination modulation.

The candidate organism or the population of candidate organisms may be selected based the presence or absence of the one or more marker-trait associations that correlate with the trait of interest. In some embodiments, the marker-trait association is a known or predicted negative association between a marker and the trait of interest. In some aspects, the method includes selecting the candidate organism or the population of candidate organisms based on the absence of a negative association.

In some embodiments, the marker-trait association is a known or predicted positive association between a marker and the trait of interest. In some aspects, the method includes selecting the candidate organism or the population of candidate organisms based on the presence of a positive association.

The recombination modulation factor may be an introduced genetic modification, a chemical recombination modulation factor, a biological recombination modulation factor, an exogenously applied recombination modulation factor, irradiation, endogenous gene activation, endogenous gene suppression, transient recombination modulation factor, or a combination thereof. In some aspects, the recombination modulation factor is a genetic modification introduced by a site-specific CRISPR-Cas system. In some aspects, the recombination modulation factor is a genetic modification introduced by a site-specific nucleobase editor without a double strand DNA break.

In some embodiments, modulated recombination may be an increase in meiotic recombination frequency or meiotic cross-over events across whole genome or a portion of the genome as compared to a control. In some embodiments, modulated recombination may be a decrease in meiotic recombination frequency or meiotic cross-over events across whole genome or a portion of the genome of the organism as compared to a control. In some aspects, modulated recombination results in reduced cross-over interference. Any method of modulating recombination may be used in the methods and compositions provided herein.

### BRIEF DESCRIPTION OF THE DRAWINGS

The disclosure can be more fully understood from the following detailed description and the accompanying drawings that form a part of this application, which are incorporated herein by reference.

FIG. 1 is a cartoon showing that the genome of a maize line is edited using genome editing technology, such as Cas9 CRISPR technology (A), the genome of a maize line is edited using Cas9 CRISPR technology to disrupt a native gene that inhibits meiotic recombination (B), the genome of a maize line is edited using Cas9 CRISPR technology to insert a gene that promotes meiotic recombination (C), and the genome of a maize line is edited using Cas9 CRISPR technology to disrupt a native gene that inhibits meiotic recombination and insert a gene that promotes meiotic recombination (D).

FIG. 2 is a cartoon showing that a genome-edited maize line from FIG. 1 is crossed with a maize line (Line A) to produce a population of maize plants that have increased meiotic recombination (Population A). Line A may be the same or different from the genome-edited maize line of FIG. 1. For example, Line A may be a maize line edited to have increased meiotic recombination, a non-modified/non-edited maize line, or a maize line edited to affect a different trait.

FIG. 3 is a is a cartoon showing that a plant from Population A of FIG. 2 is allowed to self-pollinate or is crossed with a maize line (Line B) to produce a population of maize plants that have increased meiotic recombination (Population B).

FIG. 4 is a cartoon showing an ear that has increased meiotic recombination, for example, from a maize plant of FIG. 1, FIG. 2, or FIG. 3, is pollinated. The ear may be pollinated with pollen from another plant or self-pollinated to produce a fertilized plant. For example, in one embodiment, the F1 ear is heterozygous for a knock-out of a gene that increases meiotic recombination and may be self-pollinated to produce a F2 population.

FIG. 5 is a cartoon showing an ear being pollinated with pollen that has increased meiotic recombination, for example, pollen from a maize plant of FIG. 1 FIG. 2, or FIG. 3, to produce a fertilized plant.

FIG. 6 is a cartoon showing a schematic of one embodiment of the present disclosure. (A) Kernels from a plant genome edited to have increased meiotic recombination (or derived from a progeny plant thereof) are planted and grown; (B) DNA is extracted from the plant and genotyped; (C) the plant is phenotyped for a trait of interest, for example, ear or plant height; (D) genotypic and phenotypic data are analyzed for Marker-Trait Associations (MTAs); (E) MTA's are used for Marker-Assisted Selection (MAS) to select or counter-select candidate maize lines for further use/non-use, for example, in a breeding program. The lines can be genome-edited or non-edited or modified.

FIG. 7 is a cartoon showing a new DNA combination in the offspring resulting from the homologous recombination across a substantial portion of genomic DNA from its parents, the recombination and new DNA combination are a result of using one of the methods to increase meiotic recombination described herein.

FIG. 8 is a cartoon of one embodiment of the present disclosure showing that marker-trait associations in maize lines from a population of plants that have increased meiotic recombination can be evaluated for individual genes or set of genes that contribute to or are associated with a trait of interest, e.g. shorter plant height. Lines 1 and 2 are maize lines homozygous for their respective Genes 1, 2 and 3. Due to increased meiotic recombination using the methods described herein, Gene 2 in Lines 1 and 2 undergoes homologous recombination to give rise to Lines 3 and 4. As a result, Gene 1 and SNP1 from Line 1 are no longer linked with Gene 2 or Gene 3 and SNP2 from Line 1 in Lines 3 and 4; Gene 1 and SNP3 from Line 2 are no longer linked with Gene 2 or Gene 3 and SNP4 from Line 2 in resulting Lines 3 and 4. Thus, using the methods described herein, genomic regions having one or more linked genes that impact the same trait in conflicting or differing ways may be now be observed and identified, whereas previously that genomic region containing linked Genes 1-3 may have been overlooked and disregarded for its contribution to plant height since no real impact on plant height would be observed in Lines 1 and 2. Thus, using the methods described herein, linked genes may be broken up allowing for the identification of new combinations of functional alleles, for example, for Genes 1 and 3. SNP2 and/or SNP3 associated with decreased (shorter) plant height may be used in MAS for selection of a plant with decreased (shorter) plant height and SNP1 and/or SNP4 associated with increased (taller) plant height may be used in MAS for counter-selection of a plant with increased (taller) plant height.

FIG. 9 is a flowchart depicting a typical or classical advancement process for a breeding program versus one embodiment of an advancement process for a breeding program based on the methods described herein. Top: In a classical advancement process, populations of phenotyped organisms are both genotyped and phenotyped to determine genotype-phenotype associations, so that an organism may be selected for further testing. Bottom: Using one embodiment of an advancement process described herein, populations of non-phenotyped organisms are genotyped and selected based on marker-trait associations that predict/associate with a desired trait of interest, so that an organism may be selected for further testing.

FIG. 10 is a flowchart depicting a typical or classical non-advancement process for a breeding program versus one embodiment of a non-advancement process for a breeding program based on the methods described herein. Top: In a classical non-advancement process, populations of phenotyped organisms are both genotyped and phenotyped to determine genotype-phenotype associations, so that an organism with an undesired trait may be counter-selected and/or removed from the breeding program. Bottom: Using one embodiment of a non-advancement process described herein, populations of non-phenotyped organisms are genotyped and counter-selected based on marker-trait associations that predict/associate with an undesirable trait of interest, so that an organism with an undesired trait may be counter-selected and/or removed from the breeding program.

FIG. 11 shows two bar graphs that graphically represent the quantification of maize plant height data (in cm) (A) or ear height data (in cm) (B) from progeny of F2 or F3 families as discussed in Example 1 herein.

### DESCRIPTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

Increasing recombination rates can help improve marker-trait associations by breaking linkages between genetically close markers that are less likely to be observed separately, thus increasing the resolution of MTA statistics. Increasing genetic marker density and recombination frequency within experiments establishing MTAs can help limit the chance that recombination will break the statistical associations. Advances in genomic technologies have allowed molecular markers to be placed in close proximity to all genes within the genome, ensuring that observable chromosomal variations are near variations responsible for specific phenotypes. However, the amount of recombination found in the populations within the experiment establishing the MTA tends to be more important than genetic marker density when creating marker trait associations. Higher levels of recombination among experimental entries in the initial experiment allows the position of genomic variation contributing to a phenotype to be precisely estimated with genetic markers. Traditionally, recombination has been increased within experiments by developing specific populations such as recombinant inbred lines and synthetic and nested association mapping populations. These types of populations can dramatically increase recombination frequencies but also increase the cost and time needed for population development.

Increasing recombination rates can be used for targeted trait introgression while preserving the genetic background of the targeted trait recipient. Recombination frequency can influence the application of marker trait associations even when the association is completely linked to genomic variation influencing a phenotype. Plant and animal breeding programs have developed approaches to rapidly introgress beneficial traits into elite breeding germ plasm. Introgression efficiency is directly dependent on recombination frequencies around the MTA. Lower recombination frequencies will require additional generation or larger population sizes to successfully introgress the beneficial trait while minimizing introgression of undesirable genetic material from the breeding partner carrying the beneficial trait. Lower recombination frequency can increase the introgressed chromosomal segment, introgressing the beneficial trait but also introgressing other loci contributing to undesirable phenotypic traits. Increasing recombination rates, however, allows some members of a population resulting from a cross to have the desired trait but less of the undesirable genetics.

The advent of whole genome sequencing has demonstrated that the frequency of recombination vs. chromosomal distance can change based on chromosome identity and genomic region. For example, the pericentromeric regions of the maize genome show repressed recombination, causing large chromosome segments to have very little recombination. This lack of recombination directly influences maize breeding programs where low recombination frequencies maintain unfavorable linkages between allelic variation at genes influencing commercially valuable traits. Maize breeders need to use larger populations to allow recombination to break these linkages apart to create favorable allelic combinations.

Several genes controlling recombination in plants, microorganisms, and animals have been identified. In general, these genes tend to limit recombination to ensure genome stability in plants. Some of these genes may not directly target recombination to a specific area of the genome, but rather globally increase recombination frequencies across the genome. Studies knocking-out these recombination-influencing genes have resulted in a general increase in the frequency of recombination per meiosis. Research using gene editing to modify these genes open the possibility of creating plants with higher recombination frequencies or rates. This increase in recombination could directly benefit plant breeding programs by increasing accuracy of MTA detection, increasing the precision (and thus the speed) of introgressing MTAs into elite germ plasm, and breaking up truculent, unfavorable linkages within breeding germ plasm. Disclosed herein are examples that describe how use of gene edited versions of genes influencing recombination to improve the precision of marker-trait association experiments and precisely introgress transgenic or native traits into elite breeding germ plasm. See, for example, Examples 1 and 2, provided elsewhere herein.

Disclosed herein are methods of selecting an organism with a trait of interest. The methods described herein are not to be limited to the determination of any particular trait or set of traits.

The selected organism may be a plant, mammal, insect, microorganism, or any other organism of interest. The term fungus and yeast are used interchangeably herein. As used herein, the term microorganism encompasses yeast, bacteria, and viruses. The organisms for use in the methods can be any species of the organism, including those typically used in models, for example, S. cerevisiae (yeast), Arabidopsis thaliana (plants), mouse (mammalians), and Drosophila (insects).

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, kernels, shoots, gametophytes, sporophytes, pollen, and microspores. “Progeny” comprises any subsequent generation of a plant.

Any monocot or dicot plant may used with the methods and compositions provided herein, including but not limited to a soybean, maize, sorghum, cotton, canola, sunflower, rice, wheat, sugarcane, alfalfa tobacco, barley, cassava, peanuts, millet, oil palm, potatoes, rye, or sugar beet plant. In some embodiments, the methods include a plant that is a soybean, maize, sorghum, cotton, canola, sunflower, rice, wheat, sugarcane, alfalfa tobacco, barley, cassava, peanuts, millet, oil palm, potatoes, rye, or sugar beet plant. Accordingly, any population of monocot or dicot plants may used with the methods provided herein, including but not limited to a population of soybean, maize, sorghum, cotton, canola, sunflower, rice, wheat, sugarcane, alfalfa tobacco, barley, cassava, peanuts, millet, oil palm, potatoes, rye, or sugar beet plants. In some embodiments, the genotypic data and/or phenotypic data is obtained from a population of soybean, maize, sorghum, cotton, canola, sunflower, rice, wheat, sugarcane, alfalfa tobacco, barley, cassava, peanuts, millet, oil palm, potatoes, rye, or sugar beet plants.

The data may be obtained from a population of organisms having increased meiotic recombination, such as those naturally occurring or created by human intervention. The data may be obtained from a population of organisms having an introduced genetic modification that increases meiotic recombination as compared to a control organism that does not contain the introduced genetic modification. As used herein, the term population generally refers to a plurality of organisms, for example, a population of plants means one or more plants, such as one or more maize plants.

The term recombination and meiotic recombination are used interchangeably herein. As used herein, an increase in meiotic recombination refers to any detectable increase in the rate or frequency of meiotic recombination of homologous chromosomes compared to a suitable control, for example, a cell of an organism that has not been modified to have increased meiotic recombination. Genetic recombination frequency generally refers to the probability of a crossing over event (“event”) occurring between two genetic loci. Meiotic recombination rate or frequency, such as an increase or decrease, may be determined by detecting and quantifying crossovers. Suitable techniques include but are not limited to those involved the segregation of markers and/or traits following meiosis. For example, population-wide segregation analysis of genetic makers, cytological analysis of meiocytes using microscopy, or pollen-specific fluorescent tagged lines in plants may be used to determine meiotic recombination frequency. Meiotic crossover frequency may be evaluated at the whole genome level or at specific genomic intervals. For example, crossover rate for genomic intervals may be determined with respect Centimorgan (cM)/megabase (Mb) to calculate the meiotic recombination frequency with respect to the genome size. In some embodiments, meiotic recombination is increased in a population of organisms so that the rate or frequency of meiotic recombination events is increased by more than 0.5×, 1×, 2×, 3×, 4×, 5×, 6×, 7×, 8×, 9×, 10×, 11×, 12×, 13×, 14×, 15×, 20×, 25×, or greater than the rate or frequency of meiotic recombination events in a control population of organisms or individual organism, such as a member of the population, that is not modified for increased meiotic recombination using the methods described herein. In some embodiments, the rate or frequency of meiotic recombination events is between about 0.5×-40× the rate or frequency of meiotic recombination events in a control population of organisms or individual organism, such as a member of the population, that is not modified for increased meiotic recombination using the methods described herein. For example, double haploid plants may be created that have meiotic recombination events that are increased by more than 0.5×, 1×, 2×, 3×, 4×, 5×, 6×, 7×, 8×, 9×, 10×, 11×, 12×, 13×, 14×, 15×, 20×, 25× or greater than the rate or frequency of meiotic recombination events in a control double haploid or control double haploid population not modified for increased meiotic recombination using the methods described herein.

In some embodiments, meiotic recombination is increased in a population of organisms or individual organism so that the number of crossover events is increased by more than 1, 2, 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 105, 110, 115, 120, 125, 130, 140, 150, 160, 175, 200, 250, 300, 400, or greater than the number of crossovers in a control population or organism that is not modified for increased meiotic recombination using the methods described herein. The rate or frequency of meiotic recombination or number of crossovers may be even further increased by crossing or fertilizing members of these populations with one another, with their progeny, or combinations thereof. Using this approach may reduce cross-over interference so that meiotic recombination crossovers may be observed in genomic DNA in closer proximity to one another.

“Genetic modification” generally refers to modification of any nucleic acid sequence or genetic element by insertion, deletion, or substitution of one or more nucleotides in an endogenous nucleotide sequence. Genetic modifications may be made in coding and non-coding sequences, such as promoter regions, 5′ untranslated leaders, introns, genes, 3′ untranslated regions, and other regulatory sequences or sequences that affect transcription or translation of one or more nucleic acid sequences. “Coding sequence” generally refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

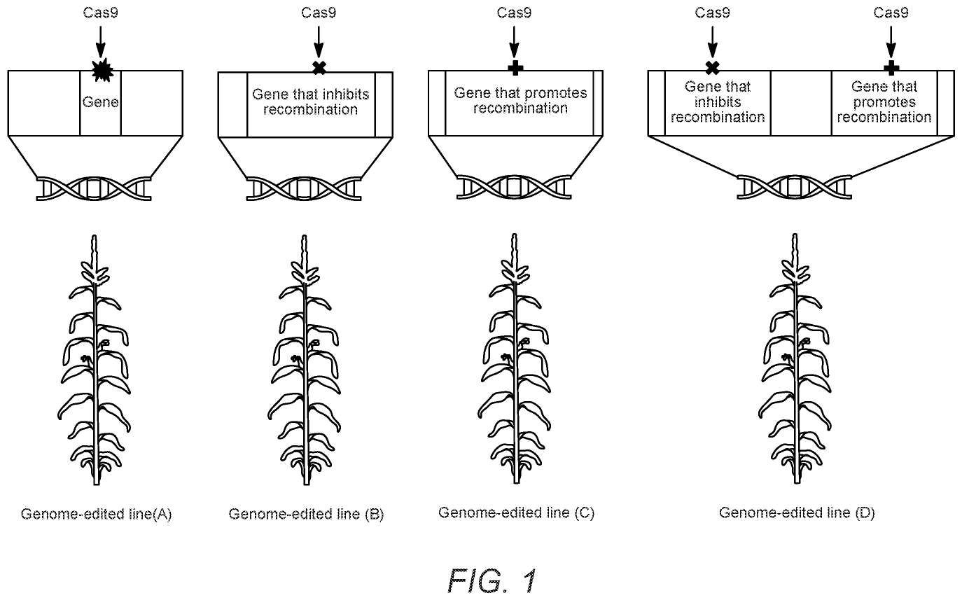

In one embodiment, through genome editing approaches described herein and those available to one of ordinary skill in the art, genes involved in inhibiting meiotic recombination and/or promoting meiotic recombination in organisms, such as plants, animals, and microorganisms may be engineered to modulate the expression of one or more host plant, microorganism, or animal endogenous genes. See, for example, FIG. 1.

The genes include but are not limited to those involved in synapsis initiation complex (SIC) or ZMM pathways, such as MSH4/MSH5 MutS-related heterodimer, MER3 DNA helicase, SHORTAGE OF CROSSOVERS1 (SHOC1) XPF nuclease, PARTING DANCERS (PTD), ZIP4/SP022, HEI10, Zip1, Zip2, Zip3, Zip4, Msh4, and Msh5, Mlh1/Mlh3, homologs thereof, orthologs thereof, or combinations thereof.

In some embodiments, the organism is modified to have increased meiotic recombination by increasing the copy number, expression level, or activity of one or more polynucleotides that promote or increase the frequency or rate of meiotic recombination, for example, those that promote crossover formation.

Provided herein are methods for increasing meiotic recombination by increasing the copy number, expression level, or activity of one or more polynucleotides that promote or increase the frequency or rate of meiotic recombination. Exemplary polynucleotides and polypeptides include but are not limited to those in the synapsis initiation complex (SIC) or ZMM pathways, such as MSH4/MSH5 MutS-related heterodimer, MER3 DNA helicase, SHORTAGE OF CROSSOVERS1 (SHOC1) XPF nuclease, PARTING DANCERS (PTD), ZIP4/SP022, HEI10, Zip1, Zip2, Zip3, Zip4, Msh4, and Msh5, Mlh1/Mlh3, homologs thereof, orthologs thereof, or combinations thereof. See, for example, Lynn et al. (2007) Chromosome Research. 15:591-605; Serra et al. (2018) PNAS. 115(10):2437-2442, each of which is herein incorporated by reference in its entirety.

In certain embodiments, methods for increasing meiotic recombination in a microorganism include increasing the copy number, expression level, or activity of one or more polynucleotides or polypeptides in the ZMM pathways including but not limited to Zip1, Zip2, Zip3, Zip4, Msh4, Msh5, Mlh1/Mlh3, Mer3, HEI10, MMS21, Shoc1/PTD homologs thereof, and orthologs thereof.

In certain embodiments, methods for increasing meiotic recombination in plants include increasing the copy number, expression level, or activity of one or more polynucleotides or polypeptides in the ZMM pathways including but not limited to, MSH4/MSH5, Mlh1/Mlh3, MutS-related heterodimer, MER3 DNA helicase, SHORTAGE OF CROSSOVERS1 (SHOC1) XPF nuclease, PARTING DANCERS (PTD), ZIP4/SP022, HEI10, homologs thereof, and orthologs thereof.

The expression level or activity of one or more polynucleotides or polypeptides that promote or increase meiotic recombination may be increased by any suitable method, for example, by increasing the copy number of the polynucleotide and/or expression level or activity of the polypeptide.

In some embodiments, the organism, including without limitation, a plant, a microorganism, or animal, is modified to have increased meiotic recombination by genetically introducing in the organism's genome one or more polynucleotides that encodes a polypeptide to increase the expression or activity of one or more genes that function to promote or increase meiotic recombination in a cell, including but not limited to HEI10, MSH4/MSH5, Mlh1/Mlh3, MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, homologs thereof, and orthologs thereof.

Provided herein are methods for increasing meiotic recombination by suppressing the expression level or activity of one or more polynucleotides that inhibit meiotic recombination. Exemplary polynucleotides and polypeptides include but are not limited to those that, alone or with other proteins, suppress homologous recombination or limit crossovers, including those in anticrossovers pathways, including, but not limited to FANCM, MHF1, MHF2, FIDGETIN-LIKE1, RECQ4, TOPOISOMERASE3a, RMI1, RMI2, RTEL1, homologs thereof, and orthologs thereof. See, for example, Serra et al. (2018) PNAS. 115(10):2437-2442, which is herein incorporated by reference in its entirety. As used herein, the term RECQ4 also includes those RECQ4 that are duplicated or present in an organism with more than one gene, for example, such as RECQ4A and RECQ4B in Arabidopsis.

In certain embodiments, methods for increasing meiotic recombination in organisms, such as plants, microorganisms, and animals, include suppressing the expression level or activity of one or more polynucleotides or polypeptides in FANCM, MHF1, MHF2, FIDGETIN-LIKE1, RECQ4, TOPOISOMERASE3a, RMI1, RMI2, and RTEL1, homologs thereof, and orthologs thereof.

In some embodiments, the activity of a FANCM, MHF1, MHF2, FIDGETIN-LIKE1, RECQ4, TOPOISOMERASE3a, RMI1, RMI2, and/or RTEL1 polypeptide, homolog thereof, and ortholog thereof is suppressed by disrupting the gene encoding the FANCM, MHF1, MHF2, FIDGETIN-LIKE1, RECQ4,TOPOISOMERASE3a, RMI1, RMI2, and/or RTEL1 polypeptide, homologs thereof, and orthologs thereof, for example, using any method known in the art, including but not limited to genome editing approaches. The organisms may be heterozygous and/or homozygous for the introduced gene edit or disruption, for example, a homozygous HEI10 knock-in and homozygous RecQ4 knock-out.

In certain embodiments, the FANCM, MHF1, MHF2, FIDGETIN-LIKE1, RECQ4, TOPOISOMERASE3a, RMI1, RMI2, and/or RTEL1 gene is disrupted by transposon tagging. In another embodiment, the FANCM, MHF1, MHF2, FIDGETIN-LIKE1, RECQ4, TOPOISOMERASE3a, RMI1, RMI2, and/or RTEL1gene is disrupted by mutagenizing organisms, such as plants or microorganisms, using random or targeted mutagenesis, such as or TUSC mutations, and selecting for organisms, e.g. plants, that have reduced FANCM, MHF1, MHF2, FIDGETIN-LIKE1, RECQ4, TOPOISOMERASE3a, RMI1, RMI2, and/or RTEL1 activity, for example, expression level, or combinations thereof. Additional methods for suppressing the expression of an endogenous FANCM, MHF1, MHF2, FIDGETIN-LIKE1, RECQ4, TOPOISOMERASE3a, RMI1, RMI2, and/or RTEL1 polypeptide in organisms, such as plants or microorganisms, may include the use of chemicals such as ethyl methanesulfonate-induced mutagenesis and deletion mutagenesis. In addition, a fast and automatable method for screening for chemically induced mutations, TILLING (Targeting Induced Local Lesions In Genomes), using denaturing HPLC or selective endonuclease digestion of selected PCR products may also be used.

In some aspects, the one or more genes that function to inhibit recombination include but are not limited to FANCM, MHF1, MHF2, FIDGETIN-LIKE1, RECQ4, TOPOISOMERASE3a, RMI1, RMI2, and/or RTEL1, homologs thereof, and orthologs thereof, or combinations thereof.

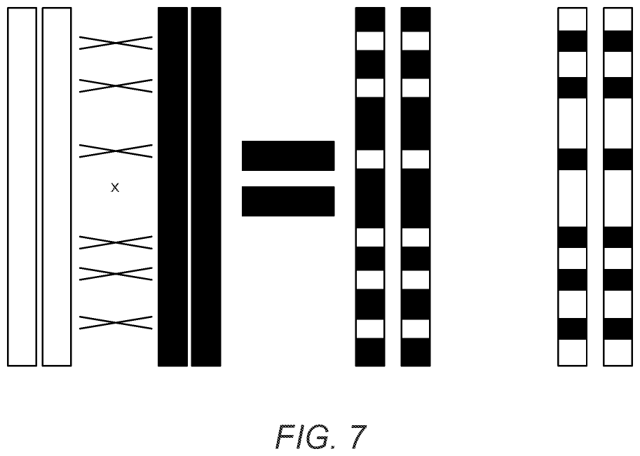

Any method of increasing recombination may be used in the methods described herein. In some embodiments, the meiotic recombination methods create random, non-specific (non-targeted) crossovers across a substantial portion of the organism's genome rather than targeting recombinations to a specific region, e.g. a centromere, telomere, pericentromere, or hotspot, genes, in the organism's genome. See, for example, FIG. 7. Although the recombination may not be targeted to a specific location within the organism's genome, homologous recombination in a specific genomic region of interest, a centromere, telomere, pericentromere, or hotspot could be evaluated for recombination using various methods and combinations described herein.

In some embodiments for increasing recombination in plants, the methods comprise increasing recombination by editing an organism's genome to suppress the activity of the gene products of one or more of: FANCM, MHF1, MHF2, FIDGETIN-LIKE1, RECQ4, TOPOISOMERASE3a, RMI1, RMI2, and/or RTEL1, homologs thereof, and orthologs thereof. In some embodiments, meiotic recombination is increased by editing the organism's genome to modify the region encoding the DUF1767 domain and/or the OB-fold domain of the RMI1 polypeptide, for example, in plants or microorganisms. In some embodiments, meiotic recombination is increased by editing the organism's genome to modify the region encoding one or more of the DEAD 2 Helicase C 2 domains of the RTEL1 polypeptide, for example, in plants or microorganisms. In some embodiments, meiotic recombination is increased by editing the organism's genome to modify one or more regions, for example, those encoding the ERCC4-like nuclease domain, the helix-hairpin-helix (HhH)₂ domain, DEXDc and/or the HELICc domain of the SF2 helicase domain in FANCM polypeptide.

In other embodiments, populations of organisms having varying levels of meiotic recombination may be used in the methods and compositions described herein, for example, those populations with increased, decreased, or non-modified meiotic recombination. In some embodiments, an organism with increased, decreased, or non-modified meiotic recombination is crossed with another organism, for example, that has increased, decreased, or non-modified meiotic recombination. The organism may come from a population of organisms. In some embodiments, the organism is modified to have decreased meiotic recombination by decreasing the copy number, expression level, or activity of one or more polynucleotides that promote or increase the frequency or rate of meiotic recombination, for example, those that promote crossover formation and/or modified to increase the copy number, expression level, or activity of one or more polynucleotides that inhibit meiotic recombination. In some embodiments, the organism, including without limitation, a plant, a microorganism, or animal, is modified to have decreased meiotic recombination by genetically introducing in the organism's genome one or more polynucleotides that encodes a polypeptide to decrease the expression or activity of one or more genes that function to promote or increase meiotic recombination in a cell, including but not limited to HEI10, MSH4/MSH5, Mlh1/Mlh3, MutS-related heterodimer, MER3 DNA helicase, SHORTAGE OF CROSSOVERS1 (SHOC1) XPF nuclease, PARTING DANCERS (PTD), ZIP4/SP022, Zip1 Zip2, Zip3, Zip4, Msh4, Msh5, Mlh1/Mlh3, homologs thereof, and orthologs thereof. In some embodiments for methods of decreasing meiotic recombination in an organism, including without limitation, a plant, a microorganism, or animal, the methods comprise decreasing recombination by editing an organism's genome to increase the expression level, activity, or copy number of the gene products of one or more of: FANCM, MHF1, MHF2, FIDGETIN-LIKE1, RECQ4, TOPOISOMERASE3a, RMI1, RMI2, RTEL1, homologs thereof, and orthologs thereof. In some embodiments, an organism having more than 1, 2, 3, 4, 5, 6, 7, 8, 9 and less than 10, 9, 8, 7, 6, 5, 4, 3, and 2 recombination events in their genome may be created from organisms with varying levels or rates of meiotic recombination, e.g. increased, decreased, or non-modified meiotic recombination. Such an organism may be used in the methods and compositions described herein, for example, to evaluate various gene interactions and/or to evaluate an individual gene's impact on epistasis.

The one or more nucleotide substitutions, additions and/or deletions may be introduced using any suitable technology or approach. In some aspects, the one or more nucleotide substitutions, additions and/or deletions is introduced using genome-editing technology. In some examples, the genome-editing technology includes an endonuclease including but not limited to Cas/CRISPR, meganuclease, zinc finger nucleases (ZFNs), or transcription activator-like effector nucleases (TALENs), or combinations thereof. In some aspects, the one or more nucleotide substitutions, additions and/or deletions is introduced using chemical mutagenesis or transposons. In some embodiments, the one or more organisms, for example, plants or animals, is modified to have increased meiotic recombination by using RNA technology to suppress the activity of one or more genes that function to inhibit meiotic recombination.

Any suitable RNA suppression technology may be used including but not limited to RNAi, microRNA, shRNA, or combinations thereof.

“Suppression DNA construct” is a recombinant DNA construct which when transformed or stably integrated into the genome of the plant, results in “silencing” of a target gene in the plant. The target gene may be endogenous or transgenic to the plant.

The terms “suppress”, “suppressed”, “suppression”, “suppressing” and “silencing”, are used interchangeably herein and include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like.

Various methods can be used to introduce one or more sequences of interest into a cell of an organism, e.g. a sequence that functions to increase meiotic recombination in the organism by increasing the expression level, copy number, or activity of polynucleotides and polypeptides that increase meiotic recombination, a sequence that functions to increase meiotic recombination in the organism by reducing the expression level, copy number, or activity of polynucleotides and polypeptides that inhibit meiotic recombination, or both. In some examples, the expression level, copy number, or activity of HEI10, MSH4/MSH5 MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, homologs thereof, or orthologs thereof is increased and the expression level, copy number, or activity of FANCM, MHF1, MHF2, FIDGETIN-LIKE1, RECQ4, TOPOISOMERASE3a, RMI1, RMI2, RTEL1, homologs thereof, or orthologs thereof is decreased in the organism. In some embodiments, HEI10 expression level, copy number, or activity is increased and RECQ4 expression level, copy number, or activity is decreased in the organism.

“Introducing” is intended to mean presenting to the organism or cell the polynucleotide or resulting polypeptide in such a manner that the sequence gains access to the interior of a cell of the organism. The methods of the disclosure do not depend on a particular method for introducing a sequence into the organism or cell, only that the polynucleotide or polypeptide gains access to the interior of at least one cell of the organism.

Genetic modifications may be introduced into a cell of the organism, such as a plant, insect, microorganism, or animal using any suitable technique or approach, for example, mutagenic chemical substances, irradiation, or genome editing. Genome editing technologies, such as meganucleases, zinc finger nucleases, transcription activator-like effector nucleases (TALENS), CRISPR Cas endonucleases (such as but not limited to Cas9), other RNA-guided endonucleases, as well as base editing technology, may also be used to introduce genetic modifications or edit the genome of an organism or a population of organisms, including plants, by genome editing or by insertion. In some examples, the genome of a population of the organisms may be edited to reduce the activity of one or more genes that function to inhibit recombination, thus increasing the meiotic recombination rate in the population.

Such Cas endonucleases include, but are not limited to Cas9 and Cpf1 endonucleases. Other Cas endonucleases and nucleotide-protein complexes that find use in the methods disclosed herein include those described in WO 2013/088446. These technologies allow for targeted modification of sequences of interest, including introducing genetic modifications into an endogenous or native host DNA sequence or a pre-existing transgenic sequence in the organism.

In some embodiments, genetic modifications may be facilitated by gene editing through the induction of a double-stranded break (DSB) in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell and in some embodiments is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800.5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148). Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012).

Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type I is endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

In addition to the double-strand break inducing agents, site-specific base conversions can also be achieved to engineer one or more nucleotide changes to create one or more EMEs described herein into the genome. These include for example, a site-specific base edit mediated by an C*G to T·A or an A·T to G*C base editing deaminase enzymes (Gaudelli et al., Programmable base editing of A·T to G*C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. Catalytically dead dCas9 fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf 1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific positions. It is understood that based on the methods and embodiments described herein utilizing a guided Cas system one can now tailor these methods such that they can utilize any guided endonuclease system.

As used herein, the term “guide polynucleotide”, relates to a polynucleotide sequence that can form a complex with a Cas endonuclease and enables the Cas endonuclease to recognize, bind to, and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof. The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, published on Feb. 26, 2015, incorporated in its entirety by reference herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. The terms “guide RNA/Cas endonuclease complex”, “guide RNA/Cas endonuclease system”, “guide RNA/Cas complex”, “guide RNA/Cas system”, “g RNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site. The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knockout, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guidepolynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site. The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.) The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 10.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

In other instances, seeds or other plant material can be treated with a mutagenic chemical substance, according to standard techniques, to introduce genetic modifications. Such chemical substances include, but are not limited to, the following: diethyl sulfate, ethylene imine, and N-nitroso-N-ethylurea. Alternatively, ionizing radiation from sources such as X-rays or gamma rays can be used to introduce genetic modifications. “TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter, for example). These mutant variants may exhibit higher or lower meiotic recombination activity than that exhibited by the gene in its natural form.

In some embodiments, the organism, such as seeds or other plant material, may be treated with a chemical inhibitor, e.g. EDTA, DMSO, and the like, an RNAi application, or wounded to increase meiotic recombination in an organism or population of organisms. See, for example, Ihkre and Kronstad. (1975) Crop Science. 15:429-431, herein incorporated by reference in its entirety.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants.

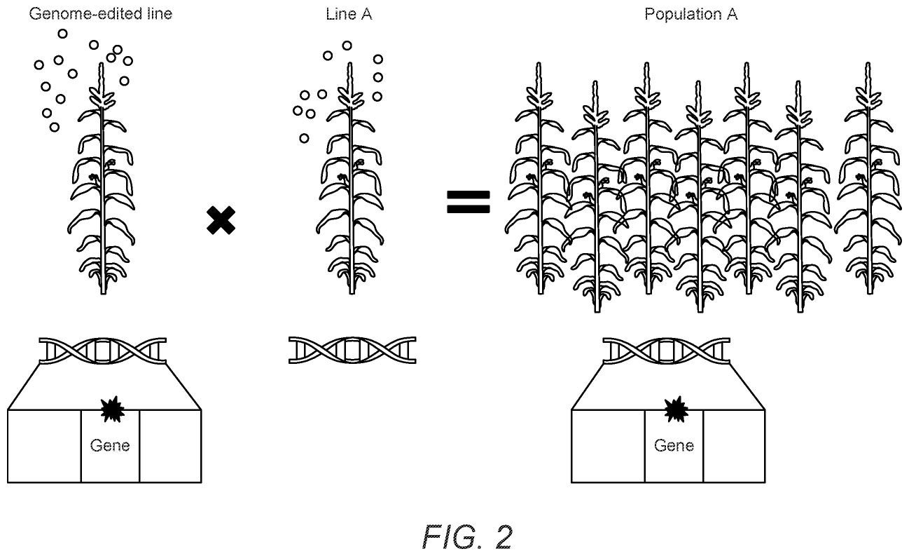

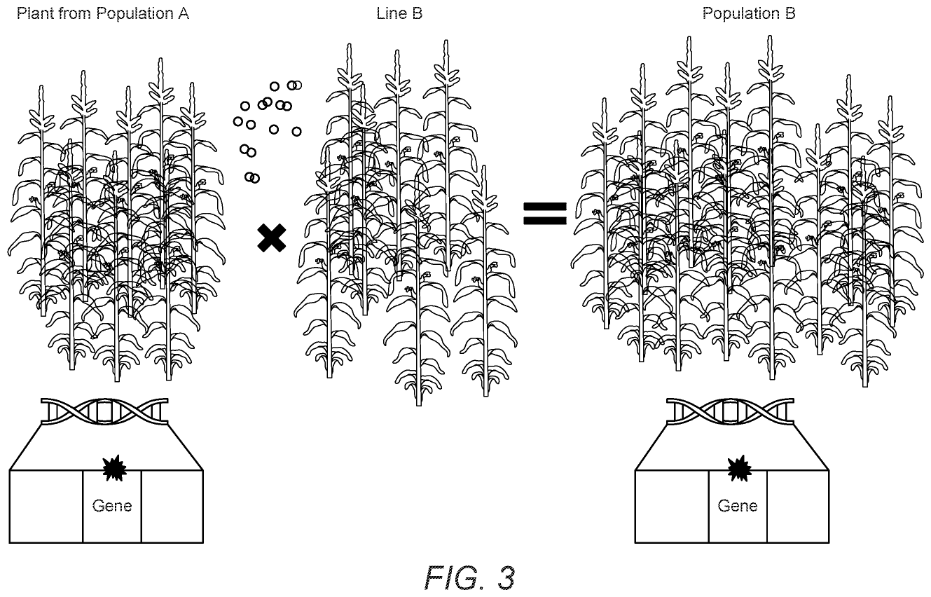

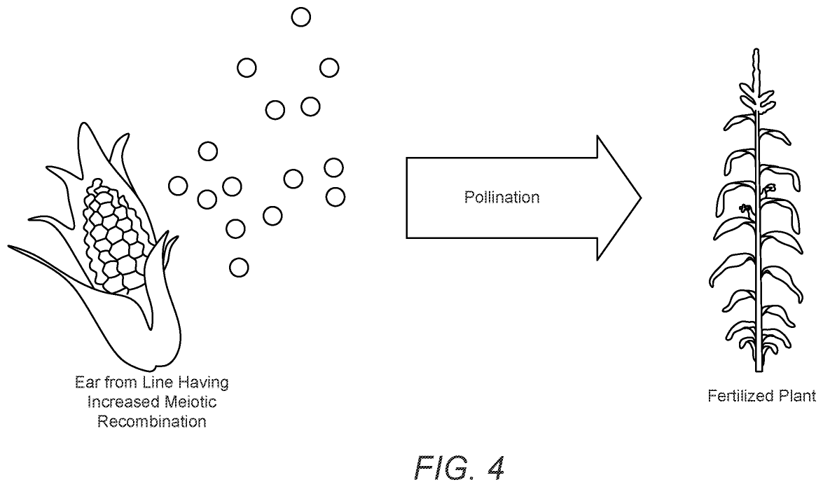

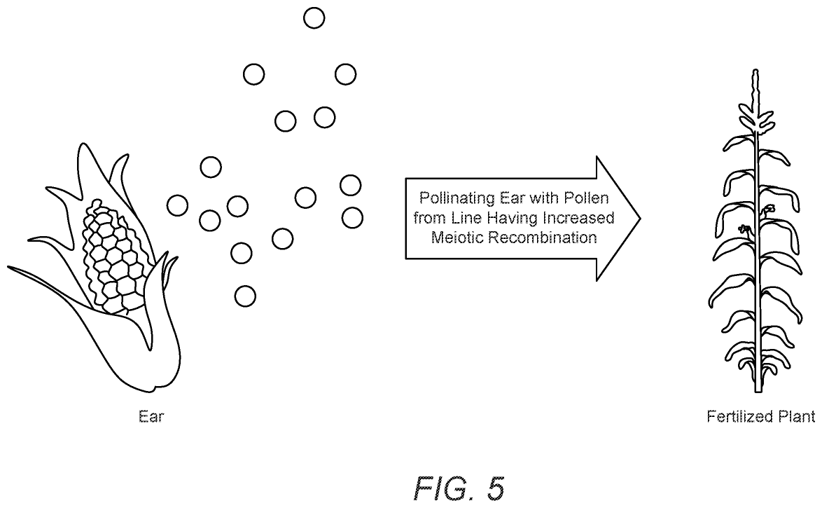

The organisms genetically modified to have increased meiotic recombination may be grown and evaluated and/or crossed using methods well known to one skilled in the art to generate populations for evaluation for change in meiotic recombination rates and/or marker-trait associations. See, for example, FIG. 2, FIG. 3, FIG. 4, and FIG. 5. In some examples, each member of the population is fertilized to generate a second generation population of offspring, which may optionally be fertilized to generate subsequent generation populations of offspring, e.g. a third generation population. Fertilization may be carried out by any suitable approach, including self-fertilization or self-pollination when the organism is plants

In some embodiments, the methods for selecting a trait of interest include providing a data set that includes genotypic data, phenotypic data, or combinations thereof. Accordingly, the organisms in the population may be genotyped, phenotyped, or both.

The genotypic and/or phenotypic data may be obtained from an existing population of organisms, those newly generated, or predicted, for example, in silico. The data may be obtained from a population of organisms having increased meiotic recombination. In some embodiments where the organism is a plant, the data set includes genotypic and/or phenotypic data from inbred plants, hybrid plants, doubled haploid plants, including but not limited to F1 or F2 doubled haploid plants, offspring or progeny thereof, or combinations thereof.

In some embodiments, the data set includes genotypic data of nucleotide variations. In some aspects, the genotypic data includes sequence information for nucleotide variations, such as single nucleotide variations, or genome-wide sequence variation. The nucleotide variation data may include but is not limited to a single nucleotide polymorphism (SNP), haplotype, simple sequence repeat (SSR), microRNA, siRNA, quantitative trait loci (QTL), transgene, deletion, mRNA, methylation pattern, or gene expression pattern, or any combinations thereof.

Any number of methods may be used to detect or determine nucleotide variation data, including but not limited to restriction fragment length polymorphisms, allele specific hybridization (ASH), amplified variable sequences, randomly amplified polymorphic DNA (RAPD), self-sustained sequence replication, simple sequence repeat (SSR), single nucleotide polymorphism (SNP), single-strand conformation polymorphisms (SSCP), amplified fragment length polymorphisms (AFLP) and isozyme markers). In some examples, each member of the population, e.g. or a subsequent generation population of offspring, such as a second or third generation, is genotyped using a set of markers associated with a specific polymorphic genomic region.

Accordingly, in some embodiments, the nucleotide variation data is Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof.

In some embodiments, the data set includes but is not limited to whole-genome or genome-wide nucleotide variation-phenotype associations.

In some embodiments, the genotypic data relates to or is expression data and includes but is not limited to data on structural variant, tissue-specific expression, gene expression, chromatin expression, chromatin accessibility, DNA methylation, histone modifications, recombination hotspots, genomic landing locations for transgenes, or transcription factor binding status, or combinations thereof.

The data set may include but is not limited to phenotypic data. In some examples, each member of the population or a subsequent generation population of offspring, such as a second or third generation of offspring, is phenotyped for a trait associated with a specific polymorphic genomic region. In some embodiments, the phenotypic data includes data on gene expression, yield, such as yield gain, grain yield, silage yield, root lodging resistance, stalk lodging resistance, brittle snap resistance, ear height, ear length, kernel rows, kernels per row, kernel size, kernel number, grain moisture, plant height, cob color, density tolerance, pod number, number of seeds per pod, maturity, time to flower, heat units to flower, days to flower, disease resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, or combinations thereof.

In some aspects, the method includes generating, identifying, or determining the association between a marker and an associated trait in an organism. One or more marker-trait associations, for example, in the data set, that correlate with the trait of interest in the population may be identified or quantified. The population may be a subsequent generation population of offspring such as a population of offspring, such as a second or third generation population of offspring.

The term “associated with” or “associated,” when referring to a nucleic acid (e.g., a genetic marker) and a trait in the context of the present disclosure, generally refers to a nucleic acid and a trait that are in linkage disequilibrium. The term “linkage disequilibrium” refers to a non-random segregation of genetic loci. This implies that such loci are in sufficient physical proximity along a length of a chromosome that they tend to segregate together with greater than random frequency. The term “genetically linked” refers to genetic loci (including genetic marker loci) that are in linkage disequilibrium and statistically determined not to assort independently. “Marker Assisted Selection” or “MAS” refers to the practice of selecting for desired phenotypes or traits among members of a breeding population using genetic markers.

The term “associated with” or “associated,” when referring to a phenotypic marker and a trait in the context of the present disclosure, generally refers to a phenotypic marker and a trait that are in linkage disequilibrium and non-random segregation of the phenotype marker with the trait in individual members of a population of organisms The correlation or association of the phenotypic marker and the trait may be statistically analyzed, for example, for statistical significance.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), RNA marker, methylation marker, a simply inherited phenotype (such as the ‘waxy’ phenotype), or phenotypic marker such as plant or seed color in soybean, starch content in maize, or eye color in fruit fly. A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects. The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide.

Markers may be defined by the type of polymorphism that they detect and also the marker technology used to detect the polymorphism. Marker types include but are not limited to, e.g., restriction fragment length polymorphisms (RFLP), isozyme markers, randomly amplified polymorphic DNA (RAPD), amplified fragment length polymorphisms (AFLPs), simple sequence repeats (SSRs), amplified variable sequences of the plant genome, self-sustained sequence replication, or single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

The association between a marker and a trait of interest may be determined for an organism as an individual or population of an organism e.g., plants, microorganisms, animals, or insects, including members or subgroups of the population.

Any marker may be used in the context of the methods and compositions presented herein to identify and/or select organisms that have the marker-trait association of interest, whether marker-trait association is newly conferred or enhanced compared to a control organism. In certain embodiments, the presence or absence of the marker-associated trait may be detected using any number of assays known in the art and described elsewhere herein and compared to a control organism that does not have the same marker-trait association.

A marker may demonstrate an initial correlation or association with a trait of interest. Marker-trait associations may additionally be updated and reevaluated as appropriate. For example, additional marker-trait associations may be identified for new markers and/or new populations, such as for newly generated germplasm or plant lines. Additionally, marker-trait associations may be reevaluated in populations having increased meiotic recombination as linkages between or among genetically close markers are broken and the statistical relationship between the markers and the trait of interest in the populations evaluated. Generally, the closer the linkage, the more useful the marker for trait selection purposes, as recombination is less likely to occur between the marker and the gene(s) correlated with the trait, which can result in false positives. In some instances, the marker is part of the gene itself, and recombination does not readily occur between the marker and the gene.

Using the methods described herein to increase meiotic recombination, QTLs or a genomic region associated with a trait of interest may be narrowed and markers in this smaller region or associated with this region evaluated for marker-trait associations. For example, the QTL may be narrowed to 30 cM, 29 cM, 28 cM, 27 cM, 26 cM, 25 cM, 24 cM, 23 cM, 22 cM, 21 cM, 20 cM, 19 cM, 18 cM, 17 cM, 16 cM, 15 cM, 14 cM, 13 cM, 12 cM, 11 cM, 10 cM, 9 cM, 8 cM, 7 cM, 6 cM, 5 cM, 4 cM, 3 cM, 2 cM, 1 cM, 0.75 cM, 0.5 cM, 0.25 cM or less as compared to the marker associated with the region in a non-modified organism or population.

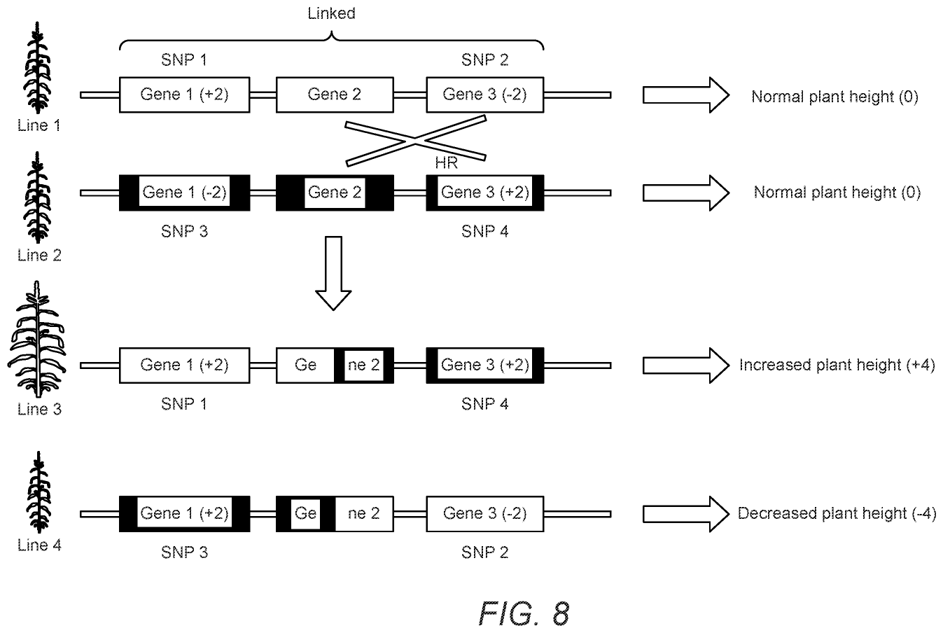

Using the same or different increased meiotic recombination populations, the association between one or markers and the trait of interest, which may be identical to or different from the marker(s) previously evaluated, may be used to identify, evaluate, confirm or unconfirm associations. See, for example, FIG. 8. The additional data may be used to update the marker-trait association information, either by replacing the marker-trait associations, or by combining the markers to generate an updated database of marker-trait associations. As such, marker-trait associations may be confirmed and updated, that is replaced and/or supplemented, as data from populations with increased meiotic recombination are obtained and evaluated. This may be a reiterative process so that marker-trait associations remain accurate and relevant for evaluation, identification, and selection purposes, for example, for selection of candidate organism(s) or improved selection of an organism with a desired trait for use in a breeding program. New or updated marker-traits associations, including allele preferences, may be entered, removed, or otherwise stored in a database for use in any of the compositions and methods described herein.

One or more organisms from the population may be selected based on marker-trait associations, for example, in certain embodiments, those genetic markers associated with certain polymorphic genomic regions and traits identified using the methods described herein. The marker-traits association data may be used to determine which candidates of a population, e.g., of plants, microorganisms, insects, or animals, are selected for breeding or counter-selected and removed from a breeding program. For example, the marker-trait association may have a negative or positive association between a marker and the trait of interest. A marker “negatively” correlates with a trait when it is linked to it and when presence of the marker is an indicator that the trait will not occur in the organism comprising the marker. A marker “positively” correlates with the trait when it is linked to it and when presence of the marker is an indicator that the desired trait will occur in an organism comprising the marker. In some instances, the marker is associated with an unfavorable trait, therefore providing the benefit of identifying candidate organisms, such as plants, microorganisms, insects, or animals, that can be counter-selected, e.g. removed from a breeding program or planting in the instance where the organism is a plant.

The marker-trait association may be determined in the increased meiotic recombination population, for example, in progeny arising from a single breeding cross, from multiple related or unrelated breeding crosses, or population of progeny selected from the breeding population at successive intervals (generations). See, for example, FIG. 2, FIG. 3, FIG. 4, and FIG. 5. In some embodiments, where the population is a population of plants, the population includes inbred plants, hybrid plants, doubled haploid plants, including but not limited to F1 or F2 doubled haploid plants, offspring or progeny thereof, or combinations thereof. In some embodiments, the plants may be heterozygous or homozygous with respect to the introduced genetic modification that increases the organism's meiotic recombination.

In some embodiments, the phenotypic data includes data on yield, such as yield gain, grain yield, silage yield, root lodging resistance, stalk lodging resistance, brittle snap resistance, ear height, ear length, kernel rows, kernels per row, kernel size, kernel number, grain moisture, plant height, density tolerance, pod number, number of seeds per pod, maturity, time to flower, heat units to flower, days to flower, disease resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, or combinations thereof.

Phenotypes or traits may be assessed by any number of techniques, including those that use the eye or an instrument or use biochemical and/or molecular means. For example, oil content, starch content, protein content, nutraceutical content, as well as their constituent components can be assessed, optionally following one or more separation or purification step, using one or more chemical or biochemical assay. Molecular phenotypes, such as metabolite profiles or expression profiles, either at the protein or RNA level, are also amenable to evaluation according to the methods described herein. For example, metabolite profiles, whether small molecule metabolites or large bio-molecules produced by a metabolic pathway, supply valuable information regarding traits of agronomic interest. Such metabolite profiles can be evaluated as direct or indirect measures of a phenotype of interest. Similarly, expression profiles can serve as indirect measures of a phenotype, or can themselves serve directly as the phenotype subject to analysis for purposes of marker correlation. Expression profiles are frequently evaluated at the level of RNA expression products, e.g., in an array format, but may also be evaluated at the protein level using antibodies or other binding proteins.

The association between the marker(s) and the trait in an organism may be identified, generated, or determined using any appropriate techniques in any suitable population. For example, the one or marker-trait associations may be identified, generated, or determined in a segregating, random or structured population. The segregation or association of the markers relative to the trait may be evaluated and the linkage or association determined using any number of methods.

A variety of methods well known in the art are available for identifying or detecting molecular markers or clusters of molecular markers that associate, i.e. co-segregate, with a trait of interest, such as those that show a statistically significant probability of co-segregation or association with a desired phenotype, manifested as linkage disequilibrium. Such methods used to detect trait loci of interest include population-based association analysis (i.e. association mapping) and traditional linkage analysis, including whole genome association analysis.

A number of statistical methods or models may be used to identify significant marker-trait associations. One such method is an interval mapping approach (Lander and Botstein, Genetics 121:185-199 (1989), in which each of many positions along a genetic map (e.g. at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

The methods may employ software programs, for example, the programs QTLCartographer® and MapQTL®, software tools such as SAS, Genstat, Matlab, Mathematica, and S-Plus, genetic modeling packages such as QU-GENE, or models such as HAPLO-MQM⁺ models.

In some embodiments, markers that have been identified as having one or more of the following: increased linkage, a significant likelihood of co-segregation, correlation, or statistical association with a trait may be used in the methods and compositions described herein. The markers may be genotypic and/or phenotypic. Using the one or more marker-trait associations that correlate with the particular trait of interest in the candidate organism, one is able to screen for and/or select a candidate organism that will exhibit the selected trait based on the detection of the presence or absence of the marker since the marker is expected to be indicative of the genotype or phenotype correlated with the trait.

As described elsewhere herein, any number of suitable techniques known to one skilled in the art may be used to detect the maker(s) in a sample of the organism's genomic DNA, for example, using RFLP, isozyme markers, RAPD, AFLP, SSRs, amplification of variable sequences of the organism genome, self-sustained sequence replication, or SNPs. SNPs can be detected e.g. via DNA sequencing, PCR-based sequence specific amplification methods, detection of polynucleotide polymorphisms by ASH, DASH, molecular beacons, microarray hybridization, oligonucleotide ligase assays, Flap endonucleases, 5′ endonucleases, primer extension, SSCP, or TGGE.

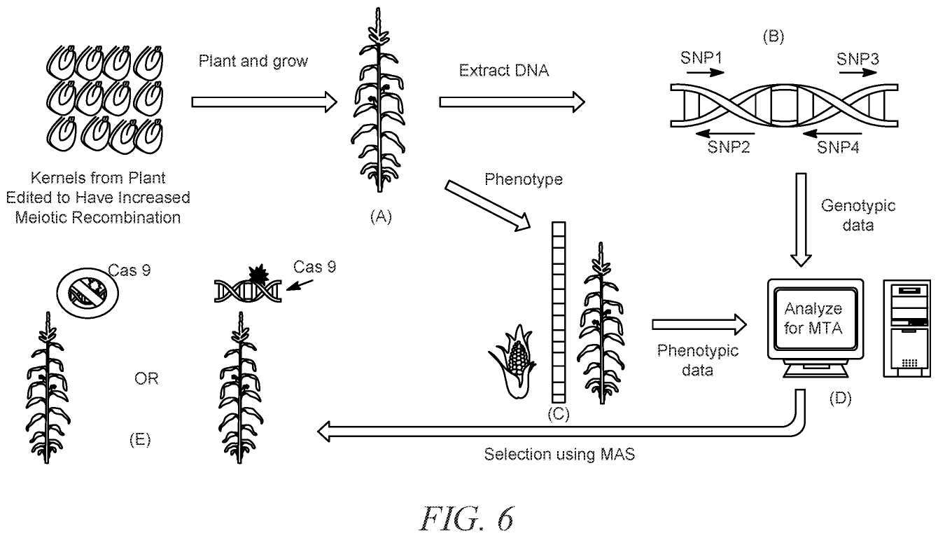

The candidate organism may be selected from a different population than initial population used to determine the marker-trait association. See, for example, FIG. 6. Indeed, the candidate organism may be selected from a population of organisms that has not been modified genetically to have increased meiotic recombination. For example, the identified or selected candidate organism having the one or more marker-trait associations may be selected from a population of non-genetically modified organisms. The candidate organism may be screened, identified, or selected from a population of candidate organisms resulting from same or different parental organisms or their progeny.

In some embodiments, the method includes screening, selecting, or identifying a candidate plant or population of candidate plants, or genotypic data and/or phenotypic data thereof from a population of candidate monocot or dicot plants, including but not limited to soybean, maize, sorghum, cotton, canola, sunflower, rice, wheat, sugarcane, alfalfa tobacco, barley, cassava, peanuts, millet, oil palm, potatoes, rye, or sugar beet plants. In some embodiments, the population of plants includes plants from doubled haploids, inbred plants, hybrid plants, or combinations thereof.

The one or more markers associated with the trait of interest may be used or extrapolated to enable marker-based selection decisions. For example, a marker or set of markers associated with a trait of interest from the database, e.g. an identical SNP from the database, may be used to screen and select or counter-select a candidate organism or population of candidate organisms from a non-genetically modified population. In some embodiments, where the identical maker, e.g. a SNP, is non-existent in the candidate organism, the candidate organism's genome may be examined for the presence of shared markers associated with the trait of interest, e.g. additional SNP or set of SNPs, and those can be used to predict the phenotype/trait for selection purposes. Additionally, or in the alternative, the absence of the identical SNP in the candidate organism may be used as basis for selection.

In some instances, the selected marker may be used as a marker for use in marker-assisted selection in a breeding program to produce organisms, such as plants, microorganisms, insects, or animals, predicted to exhibit the desired trait associated with the marker-trait association.

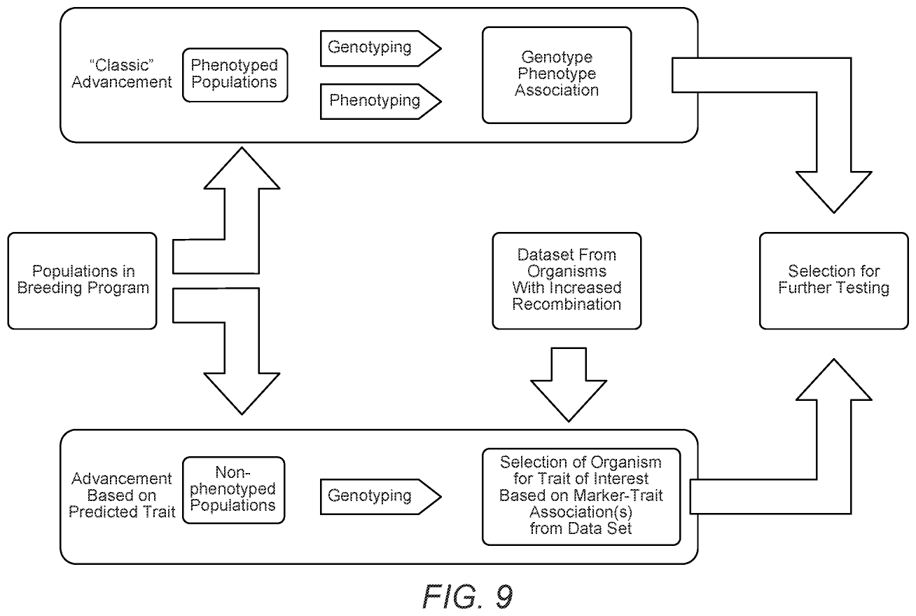

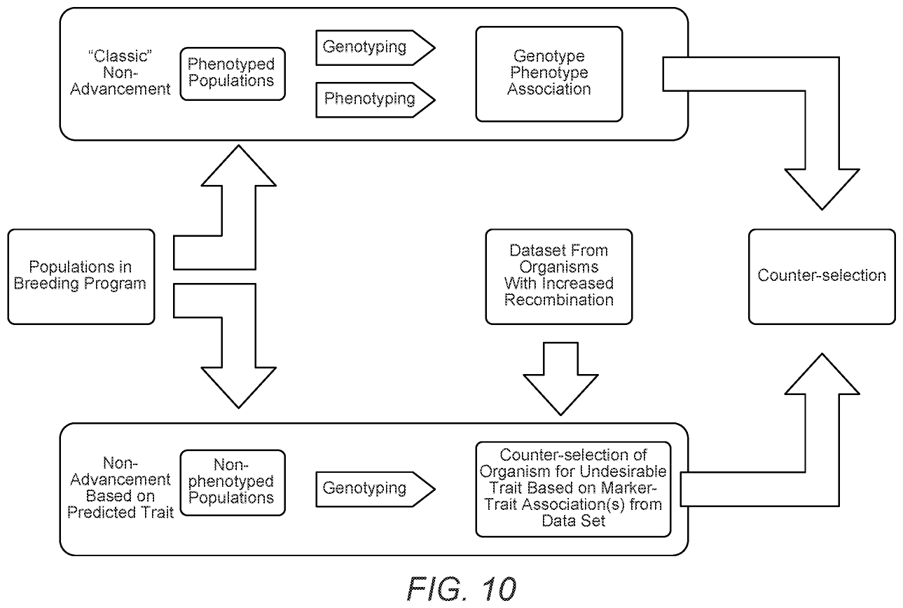

Marker-trait association data may be used to determine which candidates of a population, e.g. of plants, microorganisms, insects, or animals, are selected for breeding or counter-selected and removed from a breeding program. See, for example, FIG. 9 and FIG. 10. For example, the marker-trait association may have a negative or positive association between the marker and the trait of interest. In some instances, the marker is associated with an unfavorable trait, therefore providing the benefit of identifying candidate organisms, such as plants, microorganisms, insects, or animals, that can be counter-selected, e.g. removed from a breeding program or planting (in the case where the organism is a plant). Accordingly, organisms with an undesirable trait, e.g., such as a disease susceptible plant, may be identified, and, e.g., eliminated from certain crosses or breeding programs.

Additionally, the one or more markers associated with the trait of interest may be used in any number of marker-assisted breeding activities, for example, to screen, select and identify among new breeding populations which populations have the one or more markers, select among progeny in breeding populations progeny that have the one or more markers, and advance candidate organisms in improvement activities based on presence or absence of the one or more markers.

In some instances, the method includes using the selected candidate organism, such as the plant or animal, that that has the confirmed desired marker, e.g. marker-trait association, and/or absence of undesirable marker for use in a breeding program. For example, when the organism is a plant, the plant having the desired marker and/or absence of undesirable marker may be used in recurrent selection, bulk selection, mass selection, backcrossing, pedigree breeding, open pollination breeding, restriction fragment length polymorphism enhanced selection, genetic marker enhanced selection, double haploids, and transformation. In some instances, the plant may be crossed with another plant or back-crossed so that the marker and trait associated with it may be introgressed into the plant by sexual outcrossing or other conventional breeding methods.

The selected candidate organisms may be used in crosses to generate a population of progeny. Hence, a candidate organism containing one or more markers associated with a trait of interest is obtained and then crossed to another organism, for example, from a different population. Candidate organisms may be selected and crossed according to any breeding protocol relevant to the particular breeding program.

Accordingly, progeny may be generated from the selected candidate organism by crossing the selected organism to one or more additional organisms selected on the basis of the same marker or a different marker, e.g., a different marker for the same or a different trait of interest. In some examples, the selected candidate may be crossed to one or both parents. In the case of plants, backcrossing is usually done for the purpose of introgressing one or a few loci from a donor parent into an otherwise desirable genetic background from the recurrent parent. Introgression of a genetic trait into an organism may be carried out by any suitable approach. The term “introgression” refers to the transmission of a desired allele of a genetic locus (genetic trait) from one genetic background to another. For example, introgression of a desired allele at a specified locus can be transmitted to at least one progeny via a sexual cross between two parents of the same species, where at least one of the parents has the desired allele (genetic trait) in its genome. The desired allele may be detected by a marker that is associated with the trait. The offspring comprising the desired allele (genetic trait) can be repeatedly backcrossed to an organism, such as a line, having a desired genetic background, for example, null for the genome editing, and selected for the desired allele (genetic trait), to result in the allele becoming fixed in a selected genetic background.

In some embodiments provided herein, a genetic trait possessed by a first organism is introgressed into the genome of the offspring of a second organism that is capable of sexually reproducing with the first organism. The steps may include editing the genome of the first organism, such as a plant, to reduce the activity of one or more genes that function to inhibit meiotic recombination. The genome-edited first organism may be crossed with a second organism to generate a first population of hybrid organisms. The first population of hybrid organisms may be crossed with a second organism to generate a second population of hybrid organisms. The second population of hybrid organisms may be genotyped using markers, including a selected set of genetic markers that are within a predetermined number of bases from the genetic trait. Individuals from the second population of hybrid organisms determined to have double recombination events containing the genetic trait may be selected and used to generate a population of selected first set of individuals, which may then be crossed with a second organism to create a third population of hybrid organisms. The third population of hybrid organisms may be genotyped using any set of selected markers including one that allows for differentiation between the gene-edited genome and an unedited genome. Those individuals that have the unedited-genome and the genetic trait may be selected and crossed with the second organism to generate another population of hybrid organisms, e.g. a fourth population of hybrid organisms. The resulting population may be genotyped using the same or different set of makers or both, for example, using a set of genetic markers previously used to genotype a parent organism and genetic markers spread across the organism genome. Those individuals that have the unedited-genome, the genetic trait, and maximum or desired level genetic identity to the second organism from the population may be selected. The organism may be further backcrossed with another organism until an offspring having the genetic trait of interest becomes fixed in the desired genetic background.

The selected candidate plant may also be outcrossed, e.g., to a plant or line not present in its genealogy. Such a candidate plant may be selected from among a population subject to a prior round of analysis, or may be introduced into the breeding program de novo. The candidate plant may also be self-crossed (“selfed”) to create a true breeding line with the same genotype.

In some examples, the methods described herein include growing the candidate organism, such as a plant or animal, that has the confirmed desired marker associated with the trait of interest and/or absence of an undesired marker for further testing and evaluation.

The selected candidate organism or progeny thereof may be tested to confirm the presence or absence of the one or more marker-trait associations that correlate with the trait of interest and/or grown to confirm that the selected organism exhibits the trait associated with the marker(s). For example, in the case of plants the genotype may be confirmed and the plant grown to verify the trait. The progeny may also be evaluated genotypically using one or more of the markers as a surrogate for the marker-trait associations of interest and the progeny with the marker(s) may be selected as having the associated trait.

In certain embodiments, the presence or absence of the one or more marker-trait associations may be monitored in the candidate organism's progeny or subsequent generations from the candidate organisms, including those made in silico.

The presence or absence of the one or more markers and marker-associated trait(s) may be determined using any suitable method or technique described herein or known to one skilled in the art.

### EXAMPLES

The present disclosure is further defined in the following Examples, in which parts and percentages are by weight and degrees are Celsius, unless otherwise stated.

It should be understood that these Examples, while indicating preferred embodiments of the disclosure, are given by way of illustration only.

The disclosure of each reference set forth herein is incorporated herein by reference in its entirety.

### Example 1

Example 1 demonstrates increasing recombination through gene editing of genes regulating recombination to increase recombination in marker-trait association experiments.

The goal of this example was to develop marker-trait associations using a population of maize plants where genes suppressing recombination have been removed using gene editing approaches. The removal of these genes increased recombination, resulting in increased precision and accuracy of detected marker trait associations.

Population Development: A hybrid F1 population from a cross of two inbred parents that was homozygous for a deletion of the FANCM gene was created by CRISPR/Cas knockout as described in Example 3. Ten F1 plants were self-pollinated to create a F2 population and eight resulting F2 ears were self-pollinated to create eight F3 families. A 106 plant marker trait association experiment was created by planting 6-8 kernels from each of the eight F2 or F3 families within a greenhouse environment.

Population Genotyping: Two replicates of leaf tissue were collected from the 106 plants and DNA was extracted. The resulting DNA was genotyped at 291 SNP markers. SNP markers were selected to evenly cover known polymorphic genomic regions between the two inbred parents. Resulting discordant SNP calls between technical replicates of the same plant were removed from further analysis.

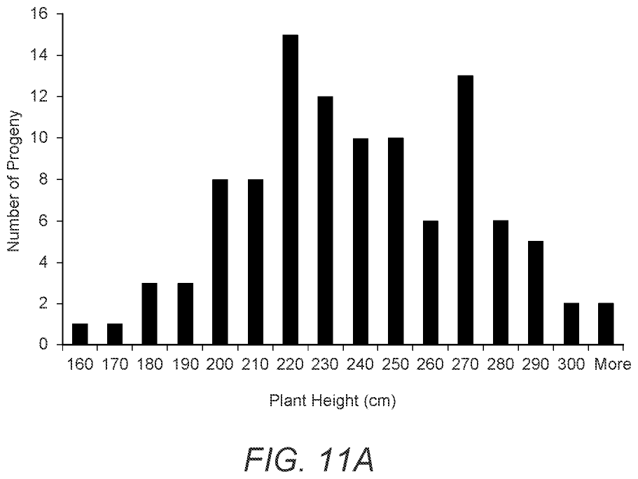

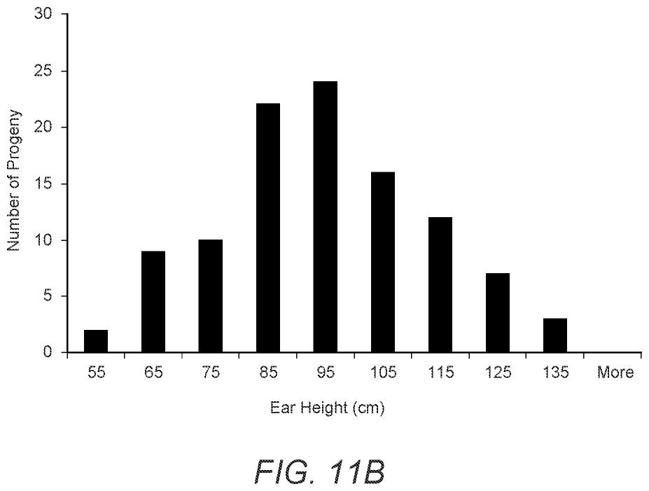

Population Phenotyping: Genotyped plants were grown to maturity and then phenotyped for plant and ear height. Plant height measurements were taken by measuring in centimeters the distance to the base of the tassel. Ear height measurement were taken by measuring the height of the base of the ear in centimeters. Due to genotyping SNP coverage issues with one plant, data for 105 plants, not 106 plants, is shown in FIG. 11. See, FIG. 11, herein for plant and ear height data.

Recombination and Quantitative Trait Locus (QTL) Analysis: Resulting genotypic and phenotypic data on the 106 plants was analyzed for marker-trait associations using the R/QTL package. Comparison of observed recombination between the F2 and F3 families showed an average increase in recombination of 6.5 recombination events. Haley-Knott regression was used to map QTLs for plant and ear height by using default parameters and using F2 family as a covariate. A permutation test using 1,000 permutations of the data was used to determine 1% likelihood thresholds for calling QTLs. SNP variation associated with lower plant and ear height was then determined at significant QTLs. One QTL for ear height and two QTLs for plant height were detected given these parameters. Increased recombination rates allowed QTLs to be precisely mapped to small intervals. The ear height QTL was mapped to a 4 cM region on maize chromosome 4 from 70.93 to 74.93 cM. Plant height QTLs were mapped to chromosome 2 118.19-126.6 cM and chromosome 5 129.82-135.1 cM respectively.

Application to Breeding Germ plasm: The ear height and plant height QTLs detected in this study were used to select for reduced plant and ear height within Pioneer Hi-Bred's commercial breeding program. Two SNP markers flanking each of the three QTLs were selected for future marker-assisted selection projects. Two double haploid populations within a 113-day maturity Pioneer breeding program previously genotyped for the selected SNPs were selected for marker-assisted selection. Individuals in these populations carrying the SNP alleles shown to be associated to increase plant and ear height were culled from the population. Remaining lines were then tested under standard breeding processes.

### Example 2

EXAMPLE 2 is a method for increasing the precision of native or transgenic trait introgression using gene edited versions of genes influencing recombination.

This example describes how gene editing of genes suppressing recombination frequency can be used to increase the frequency and precision of recombination events around key native and transgenic genes. This increase of precision can directly augment the efficiency of native and transgenic trait integration.

Development of Precise Recombinants: A donor inbred or variety (referred to here as the donor line) carrying beneficially allelic variation at a commercially valuable native or transgenic gene is selected for gene editing using CRISPR/Cas approaches. Key genes suppressing recombination are excised from the genome of the donor line using CRISPR/Cas approaches. A backcross population is created by crossing the gene edited donor line to an elite breeding line, inbred, or variety (referred to here as the elite line) and then crossing the resulting F1 population again to the elite breeding line. 1,536 individuals from the resulting backcross population is genotyped using 4 SNPs falling within 5-10 kilobases (kb) of the gene carrying beneficial native or transgenic allelic variations. Resulting genotypic data at these SNPs is used to select individuals containing double recombination events containing the beneficial allele at the native gene.

Development of Elite Line Carrying Beneficial Allele: Individuals containing double recombinants around the targeted native or transgenic gene are crossed to the elite breeding line. 1,536 individuals from the resulting population are genotyped using 4 SNPs falling within 5-10 kb of the gene suppressing recombination, 4 SNPs falling within 2 kb of the native/transgenic gene, and a SNP to differentiate the gene edited from the wild-type allele of the gene. Individuals carrying the wild-type allele at the recombination suppressing gene and the beneficial allele at the native/transgenic gene are selected and then crossed again to the elite breeding line. 1,536 individuals from the resulting population are genotyped with 3,000 SNPs spread evenly across the genome as well as 4 SNPs within 2 kb of the native/transgenic gene target and the gene suppressing recombination. A single individual carrying the beneficial allele at the native/transgenic gene, the wild-type allele at the gene suppressing recombination, and that have the maximum genetic similarity to the elite breeding line are self-pollinated. The resulting population is submitted for three more rounds of identical genotyping, selection, and self-pollination using the same SNP panel to develop a single line that is homozygous for the beneficial allele at the native gene, homozygous for the wild-type allele at the gene suppressing recombination, and maximizes genome similarity to elite breeding line.

### Example 3

EXAMPLE 3 is the method used to generate the FANCM knockout mutant plants used in Example 1.

Maize embryos of a maize hybrid were edited to alter function of the native FANCM gene. Editing was accomplished using standard transformation methods of CRISPR/Cas9 bombardment in conjunction with the use of a single guide RNA (fancm-CR4). The guide RNA target sequence and FANCM gene model are in Table 1.

TABLE 1
Guide RNA target sequence for FANCM knockout
edit.

| Guide RNA name | Guide RNA target sequence |
|---|---|
| fancm-CR4 | gatgaggctcatcgagcgtc |

Amplicon sequencing of the intended target site identified a desired edit in the T0 mutant line. Results from the DNA sequence analysis are provided in Table 2. Included in the results are the wild type sequence, and the sequence obtained from the edited line, allele 1, a description of the allele mutation and the resulting amino acid sequence of the wild type and edited allele product.

TABLE 2
DNA Sequence Analysis Showing Edit of FANCM Target Site.

| | | | AA sequence |
|---|---|---|---|
| | | | at the |
| | Allele | NT | fancm_CR4 |
| | mutation | sequence at the fancm_C4 target | target |
| Wildtype | — | TGCATGGTACAACAAATAGTTTGCTTAGTG | VIDEAHRASG |
| | | ATAGATGAGGCTCATCGAGCGTCAGGAAA | NYAYCM |
| | | TTATGCATACTGCATGGTTATCCGAGAGG | |
| | | TATGTTTCACTGTCATGTACTACACTCATT | |
| | | TGTTTTACCTGCAACACTACCGTCGGATC | |
| | | GT | |
| T0 edited | 9 bp | TGCATGGTACAACAAATAGTTTGCTTAGTG | VIDEA--- |
| line allele 1 | deletion | ATAGATGAGGCGTCAGGAAATTATGCATA | SGNYAYCM |
| | | CTGCATGGTTATCCGAGAGGTATGTTTCA | |
| | | CTGTCATGTACTACACTCATTTGTTTTACC | |
| | | TGCAACACTACCGTCGGATCGTGCGTGTA | |
| | | TAA | |

Phenotype of maize FANCM mutant. The T0 edited line was carried out to a maize inbred line. The T0 male backcross produced T0 seed, which was grown, sampled, and genotyped using Taqman SNP genotyping. A total of 80 backcross plant tissue samples were genotyped using 263 SNP markers spanning all 10 maize chromosomes. SNP data for wildtype and edited plants were used to generate additive linkage maps. Maize lines harboring FANCM edits exhibited up to two-fold increase in recombination over wildtype materials. The cumulative genetic distance increased from 1856.2 cM in the wildtype (unedited) plants to 3841.8 cM in FANCM mutant background.

### Examples 4-7

The following examples provide alternative methods for increasing the rate of meiotic recombination in maize.

### Example 4

Increase Meiotic Recombination in Maize by Modifying the c-Terminal Ob2 Domain of ZmRMI1

RMI1 stands for “RecA Mediated Instability 1”. In yeast, Sgs1-Top3-Rmi1 is a major NCO (non-crossover) promoting factor, and RecQ4 is the plant homolog for yeast Sgs1. A point mutation (Atrmil-G592×), disrupting OB2 domain of AtRmi1 gene, can enhance meiotic recombination rate up to 430% in Arabidopsis (Seguela-Arnaud et al, 2017). Rmi1 KO (knockout) will lead to male sterility in plants because Rmi1 may also play important roles to resolve meiotic recombination intermediates other than its anti-crossover function. Therefore, in this example, the N-terminal function of Rmi1 gene was preserved by only modifying the C-terminal OB2 domain.

In this study, a CRISPR/Cas9 system was used to modify the OB2 domain of the ZmRMI1 gene. ZM-RMI1-CR1 is the targeting exon 6 of ZmRMI1, and ZM-RMI1-CR2 is the targeting exon 7 of the same gene (Table 3). Both gRNAs targeted the OB2 domain of ZmRMI1 gene. A T-DNA vector was built to contain a Cas9 expression cassette and two gRNAs (ZM-RMI1-CR1+ZM-RMI1-CR2). The T-DNA vector was directly delivered into a first set of hybrid embryos from a cross of two inbred maize lines via agrobacterium-mediated transformation. T0 plants with bi-allelic dropouts or frameshift mutations at the OB2 domain were identified by direct sequencing. In addition, one T-DNA vector containing a Cas9 expression cassette alone (no gRNA) was also transformed to a second set of hybrid embryos separately. In this case, all T0 plants from the transformation of the second set of hybrid embryos were expected to have only wild-type ZmRMI1 alleles, and thus to serve as a background control for meiotic recombination.

T0 plants are grown into maturity in a greenhouse, and crossed with one of the parent inbred lines. T0 seeds are harvested and germinated, and genomic DNAs are extracted from T1 seedlings. Taqman probes from Chromosome V are used to measure the rates of meiotic crossovers. Genetic assays are used to determine the degree to which disruption of the OB2 domain of ZmRMI1 increase the rates of meiotic crossovers in maize. Phenotypic observations are also conducted to determine correlations between OB2 domain modification and male sterility.

TABLE 3
The gRNA sequences for ZmRMI1 gene.

| gRNA | gRNA sequences | Target |
|---|---|---|
| ZM-RMI1- | GTATACATAAAGCTCGTACT | gRNA that targets maize genomic |
| CR1 | | sequence |
| | | ATATACATAAAGCTCGTACTtGG in exon |
| | | 6 of ZmRMI1 gene |
| ZM-RMI1- | GGTGCAGCAGTAACCTCTCC | gRNA that targets maize genomic |
| CR2 | | sequence |
| | | AGTGCAGCAGTAACCTCTCCaGG in |
| | | exon 7 of ZmRMI1 |

### Example 5

Increase meiotic recombination in maize by knocking out ZmRMI2 gene.

RMI2 stands for “RecA Mediated Instability 2”. In humans, RMI2 physically interacts with C-terminal OB2 domain of RMI1 (Wang et al, 2010). RMI2 has been shown to slightly suppress somatic homologous recombination in Arabidopsis (Rohrig et al, 2016). It is still unknown whether RMI2 plays any role in the regulation of meiotic recombination. The physical interaction between RMI2 and the C-terminal OB2 domain of RMI1 may play a role in regulating meiotic crossovers.

CRISPR/Cas9 technology was used to knock out the ZmRMI12 gene in a first and second set of hybrid embryos, each from a crosses of the same two inbred maize lines in order to determine the extent to which a ZmRMI2 KO increased meiotic crossovers in maize. Three gRNAs were designed to target the ZmRMI2 gene (Table 4). Among them, ZM-RMI2-CR1 and ZM-RMI2-CR2 target upstream or downstream of ZmRMI2 ORF, and the purpose was to drop out the intact ORF of ZmRMI2 gene. ZM-RMI2-CR3 was targeting exon II of ZmRMI2, and the major goal was to produce frameshift mutations in order to disrupt the early translation of ZmRMI2 protein. Two T-DNA vectors were built to knock out ZmRMI2 via agrobacterium-mediated transformation. A first T-DNA vector with a Cas9 expression cassette included two gRNAs (ZM-RMI2-CR1 and ZM-RMI2-CR2), whereas a second T-DNA vector with a Cas9 expression cassette included only one gRNA (ZM-RMI2-CR3). The first T-DNA vector was delivered into the first set of hybrid embryos and the second T-DNA vector was delivered into the second set of hybrid embryos, both via agrobacterium-mediated transformation. T0 plants with bi-allelic dropouts or frameshift mutations at ZmRMI2 gene were identified by direct sequencing. In addition, T0 plants derived from the transformation of the second set of hybrid embryos described in the previous example (Example 4) serves as a background control for meiotic recombination rate. A similar assay described in Example 4 can be used to check the rates of meiotic recombination in ZmRMI2 mutants and control plants.

TABLE 4
The gRNA sequences for ZmRMI2 gene

| gRNA | gRNA sequences | Target |
|---|---|---|
| ZM-RMI2- | GCGAATTTACGGCCCGAGA | Target maize genomic sequence |
| CR1 | G | GCGAATTTACGGCCCGAGAGcG |
| | | G upstream of ZmRMI2 |
| ZM-RMI2- | GCCTAAATATTTAGTGATCC | Target maize genomic sequence |
| CR2 | | TCCTAAATATTTAGTGATCCtGG |
| | | in 3' UTR of ZmRMI2 |
| ZM-RMI2- | GCGTACCTGGCCGCCAGAA | Target maize genomic sequence |
| CR3 | C | GCGTACCTGGCCGCCAGAACcG |
| | | G in exon 2 of ZmRMI2 |

### Example 6

Increase Meiotic Recombination in Maize by Knocking Out ZmRTEL1 Gene

RTEL1 stands for “Regulator of Telomere Elongation Helicase 1”. RTEL1 homolog is present in human and plants, but absent from yeast. AtRTEL1 has a much stronger anti-recombination activity in mitosis than AtFANCM gene (Recker et al, 2014). However, it is not clear whether RTEL1 gene has a similar anti-recombination effect in meiosis.

CRISPR/Cas9 technology is used to knock out ZmRTEL1 gene in hybrid embryos from a cross of two inbred lines. Three gRNAs were designed to target ZmRTEL1 (Table 5). Among them, ZM-RTEL1-CR1 and ZM-RTEL1-CR2 are targeting upstream or downstream of ZmRMI2 ORF to delete the intact ORF of ZmRTEL1 gene. The ZM-RTEL1-CR3 is targeting exon II of ZmRTEL1 to produce frameshift mutations, which can disrupt the translation of ZmRTEL1. Two T-DNA vectors were built to knock out ZmRTEL1 via agrobacterium-mediated transformation. The first T-DNA vector contains Cas9 expression cassette with two gRNAs (ZM-RTEL1-CR1 and ZM-RTEL1-CR2), whereas the second T-DNA vector contains Cas9 expression cassette with only one gRNA (ZM-RTEL1-CR3). Both T-DNA vectors were separately delivered into hybrid embryos from the crossed inbred lines via agrobacterium-mediated transformation. T0 plants with bi-allelic dropouts or frameshift mutations at ZmRTEL1 gene were identified by direct sequencing. T0 plants from the transformation of the second set of hybrid embryos in Example 4, expected to have only wild-type ZmRMI1 alleles, and thus to serve as a background control for meiotic recombination rate. A similar assay described in Example 4 will be used to check the rates of meiotic recombination in ZmRTEL1 mutants and control plants.

TABLE 5
The gRNA sequences for ZmRTEL1 gene.

| gRNA | gRNA sequences | Target |
|---|---|---|
| ZM- | GTTGCAACGTGTCAATCAAG | Target maize genomic sequence |
| RTEL1- | | TTTGCAACGTGTCAATCAAGCG |
| CR1 | | G upstream of ZmRTEL1 |
| ZM- | GCAAGAATCTGCTAATGTTC | Target maize genomic sequence |
| RTEL1- | | CCAAGAATCTGCTAATGTTCCG |
| CR2 | | G in the 3′ UTR of ZmRTEL1 |
| ZM- | GCTGGAGAGTCCTACGGGTA | Target maize genomic sequence |
| RTEL1- | | GCTGGAGAGTCCTACGGGTAC |
| CR3 | | GG in exon 2 of ZmRTEL1 |

### Example 7

Increase Meiotic Recombination in Maize by Knocking Out ZmRecQ4

Sgs1 (Slow Growth Suppressor 1) is a RecQ family DNA helicase from yeast. Rates of meiotic crossovers in Sgs1 mutant increase 1.4 fold compared to wild-type control (Rockmill et al 2003). BLM helicase from human is an ortholog of yeast Sgs1. Hyper somatic crossover has been observed from somatic cells of person with Bloom's syndrome (Langlois et al, 1989). RecQ4 is the ortholog of Sgs1 and BLM helicase. Arabidopsis has two RecQ4 genes in the genome: AtRecQ4A and AtRecQ4B. Mutations of both genes lead to six-fold increase of meiotic crossover rates compared to wild-type controls (Seguela-Arnaud et al, 2015). There is only one single copy RecQ4 in the maize genome, and the following study is designed to test if ZmRecQ4 KO can enhance meiotic recombination in maize.

A CRISP R/Cas9 system is used to knock out the ZmRecQ4 gene in hybrid embryos from a cross between two inbreds. Two pairs of gRNAs were designed to induce dropout deletions for the ZmRecQ4 gene (Table 6). The recQ4-CR2 and recQ4-CR5 guide RNAs target 5′ UTR and exon 13 of ZmRecQ4, respectively. The recQ4-CR4 and recQ4-CR6 target exon 10 and exon 15 of ZmRecQ4, respectively. For the first experiment, immature hybrid embryos were bombarded with a Cas9 expression cassette and two gRNA expression vectors (recQ4-CR2 and recQ4-CR5). For the second experiment, immature hybrid embryos were bombarded with a Cas9 expression cassette and two gRNA expression plasm ids (recQ4-CR4 and recQ4-CR6). Three T0 plants with bi-allelic 1.8-kb dropouts at ZmRecQ4 were identified by PCR amplification and direct sequencing. In addition, T0 plants with only wild-type ZmRecQ4 allele were also identified as background controls for meiotic recombination.

TABLE 6
The gRNA sequences for ZmRecQ4 gene

| | | | | Potential |
|---|---|---|---|---|
| gRNA | | | Targeted | deletion |
| pair | gRNA | gRNA sequences | region | (bp) |
| #1 | recq4-CR2 | GGATTCCGCGGAAATGGGTG | 5′ UTR | 5886 |
| | recq4-CR5 | GCTACAGTTGCATTTGGGA | Exon 13 | |
| #2 | recq4-CR4 | GCTCATTGTGTAAGCCAGTG | Exon 10 | 1784 |
| | recq4-CR6 | GTGGACGTGCGGGTAGAGAT | Exon 15 | |

T0 plants grew into maturity in a greenhouse, and were crossed with one of the parent inbred lines. T0 seeds were harvested and germinated, and genomic DNA was extracted from T1 seedlings. Progeny (on average 100 T1 plants per T0) from T0 plants with dropout deletions and two wild-type T0 were selected for TGBS analysis. In the end, 385 informative markers were used to analyze the rates of meiotic crossovers. On average, around 21 crossovers per gamete were observed in wild-type controls. In contrast, around 62 crossovers per gamete were observed in ZmRecQ4 KOs. The cumulative genetic distance increased from 2181 cM in wild-type control to 9452 cM in ZmRecQ4 mutants (Table 7). In addition, ZmRecQ4 dropout deletions were generated in both parent inbred lines, then these edited inbreds were crossed. Taqman markers (238) were used to analyze the effect of ZmRecQ4 KO on meiotic crossovers, and a similar increase of meiotic recombination was found in the ZmRecQ4 mutants.

TABLE 7
Cumulative genetic distance in ZmRecQ4 mutants

| BC1F1 SIID | Genotype | Additive distance (cM) |
|---|---|---|
| 90277326 | WT-1 | 2087.18 |
| 90220119 | WT-2 | 2277.76 |
| 90308544 | Recq4 KO-1 | 9679.5 |
| 90341756 | Recq4 KO-2 | 9361.19 |
| 90341733 | Recq4 KO-3 | 9318.06 |

While this invention has been discussed in terms of various embodiments and examples, those of ordinary skill in the art will recognize that the invention is not limited to those particular embodiments and examples. For example, increases in recombination rates have been demonstrated using CRISPR/Cas9 gene editing tools, but similarly effective edits may be made using any gene editing tools known to those of ordinary skill in the art, including, for example, using zinc finger nucleases (ZFNs) or transcription activator-like effector nucleases (TALENs). Alternatively, increased recombination rates may be achieved by mutating repair genes by natural mutations, mutagenesis, or transposons, for example.

Furthermore, while the examples focused on increasing recombination in maize plants, those of ordinary skill in the art would recognize the benefits of increased recombination as disclosed in this specification in any plant or animal breeding program. Plant breeding programs that could benefit from the disclosed invention include: soy, maize, sorghum, cotton, canola, sunflower, rice, wheat, sugarcane, tobacco, barley, cassava, peanuts, millet, oil palm, potatoes, rye, sugar beets, and food, feed, and oil fruits, vegetables, and seeds/pods.

1. A method of selecting a plant with a trait of interest, the method comprising:
providing a data set comprising genotypic and/or phenotypic data obtained from a population of plants, wherein one or more plants in the population comprise one or more introduced genetic modifications that increase meiotic recombination in one or more plants as compared to a control plant that does not comprise the one or more introduced genetic modifications, and wherein the population of plants comprises one or more phenotypic or genotypic markers;
identifying or generating one or more marker-trait associations in the data set that correlate with the trait of interest in the population of plants;
screening a candidate plant or a population of candidate plants for the presence or absence of the one or more marker-trait associations that correlate with the trait of interest, wherein the candidate plant or the population of candidate plants (i) do not comprise the introduced genetic modifications and (ii) are not obtained from the population of plants that contain the introduced genetic modifications; and
selecting the candidate plant based on the presence or absence of the one or more marker-trait associations that correlate with the trait of interest.

2. (canceled)

3. (canceled)

4. The method of claim 1, wherein the one or more marker-trait associations is newly conferred.

5. The method of claim 1, wherein the one or more marker-trait associations has increased statistical association as compared to the corresponding marker-trait association in a control plant.

6. The method of claim 1, further comprising: growing the selected candidate plant.

7. The method of claim 1, wherein the data set comprises nucleotide variation data, phenotypic data, or combinations thereof.

8. (canceled)

9. The method of claim 1, wherein the data set comprises genome wide nucleotide variation-phenotype associations.

10. (canceled)

11. The method of claim 1, wherein the marker-trait association is a known or predicted negative association between the marker and the trait of interest.

12. The method of claim 1, wherein the marker-trait association is a known or predicted positive association between the marker and the trait of interest.

13. The method of claim 11, further comprising: selecting the candidate plant based on the absence of a negative association.

14. The method of claim 12, further comprising: selecting the candidate plant based on the presence of a positive association.

15. The method of claim 1, wherein the data set comprises genotypic and/or phenotypic data from doubled haploid plants, inbred, hybrid plants, offspring thereof, or combinations thereof.

16. The method of claim 1, wherein the population of plants is modified to have increased meiotic recombination by genetically introducing one or more polynucleotides in the plant's genome to increase the expression level or activity of one or more genes that function to promote meiotic recombination.

17. The method of claim 16, wherein the one or more genes that function to promote meiotic recombination comprises HEI10, MSH4/MSH5, Mlh1/Mlh3, MutS-related heterodimer, MER3 DNA helicase, SHORTAGE OF CROSSOVERS1 (SHOC1) XPF nuclease, PARTING DANCERS (PTD), ZIP4/SP022, Zip1, Zip2, Zip3, Zip4, Msh4, Msh5, Mlh1/Mlh3, or combinations thereof.

18. The method of claim 1, wherein the population of plants is modified to have increased meiotic recombination by genetically introducing one or more nucleotide substitutions, additions and/or deletions in the plant's genome to reduce the expression level or activity of one or more genes that function to inhibit meiotic recombination.

19. The method of claim 18, wherein the one or more genes that function to inhibit meiotic recombination comprises RMI1, RMI2, RTEL1, RECQ4, or FANCM, or combinations thereof.

20. The method of claim 1, wherein the genetic modification is introduced using genome-editing technology.

21. The method of claim 1, wherein the candidate plant or population of candidate plants comprises doubled haploid plants, inbreds, hybrid plants, offspring thereof, or combinations thereof.

22. The method of claim 1, wherein the plants in the population of plants are monocot or dicot plants.

23. (canceled)

24. (canceled)

25. (canceled)

26. (canceled)

27. (canceled)

28. A method of selecting an organism with a trait of interest, the method comprising:
providing a data set comprising genotypic and/or phenotypic data obtained from a population of organisms, wherein one or more organisms in the population comprise one or more introduced genetic modifications that increase meiotic recombination in the one or more organisms as compared to a control organism that does not comprise the one or more introduced genetic modifications, and wherein the population of organisms comprises one or more phenotypic or genotypic markers;
identifying or generating one or more marker-trait associations in the data set that correlate with the trait of interest in the population of organisms;
screening a candidate organism or a population of candidate organisms for the presence or absence of the one or more marker-trait associations that correlate with the trait of interest, wherein the candidate organism or the population of candidate organisms (i) do not contain said introduced genetic modifications and (ii) are not obtained from the population of organisms that contained said introduced genetic modifications; and
selecting the candidate organism or population of candidate organisms based the presence or absence of the one or more marker-trait associations that correlate with the trait of interest.

29. The method of claim 28, wherein meiotic recombination is increased across the genome of the organism.

30. The method of claim 28, wherein the candidate organism or population of candidate organisms is a plant, animal, or microorganism.

31. The method of claim 30, wherein the wherein the candidate organism is a plant and the population of plants comprises doubled haploid plants, inbred, hybrid plants, offspring thereof, or combinations thereof.

32. The method of claim 28, further comprising: growing the selected candidate organism.

33. A method for increasing the association between a genetic marker and an associated genetic trait in an organism comprising:
a. editing the genome of one or more members of a population of the organism to modulate the activity of one or more genes involved in recombination during meiosis, thereby increasing the meiotic recombination rate in the population;
b. fertilizing each member of the population to generate a second generation population of offspring;
c. genotyping each member of the second generation population of offspring using a set of markers associated with a polymorphic genomic region;
d. phenotyping each member of the second generation population of offspring for a trait associated with the polymorphic genomic region; and
e. quantifying one or more marker-trait associations across the second generation population of offspring.

34. The method of claim 33, wherein the organism is a plant.

35. The method of claim 33, wherein one or more marker-trait associations has increased statistical association as compared to the corresponding marker-trait association in a control organism.

36. A method of selecting a plant with a trait of interest or selecting a plant with a desired genotype, the method comprising:
providing a data set comprising genotypic and/or phenotypic data obtained from a population of plants, wherein one or more plants in the population (i) exhibit a modulated recombination pattern as compared to a control plant due to a recombination modulation factor or (ii) are progeny of one or more parental plants that exhibit modulated meiotic recombination due to a recombination modulation factor, as compared to a control plant, and wherein the population of plants comprises one or more phenotypic or genotypic markers;
identifying or generating one or more marker-trait associations in the data set that correlate with the trait of interest or the desired genotype in the population of plants;
screening a candidate plant or a population of candidate plants for the presence or absence of the one or more marker-trait associations that correlate with the trait of interest, wherein the candidate plant or the population of candidate plants (i) do not comprise the modulated recombination pattern due to the modulation factor and (ii) are not the progeny of parental plants that exhibited modulated meiotic recombination due to a recombination modulation; and
selecting the candidate plant based on the presence or absence of the one or more marker-trait associations that correlate with the trait of interest.

37. The method of claim of claim 36, wherein the recombination modulation factor is selected from the group consisting of introduced genetic modification, a chemical recombination modulation factor, a biological recombination modulation factor, an exogenously applied recombination modulation factor, irradiation, endogenous gene activation, endogenous gene suppression, transient recombination modulation factor, and a combination thereof.

38. The method of claim 36, wherein the recombination modulation factor is a genetic modification introduced by a site-specific CRISPR-Cas system.

39. The method of claim 36, wherein the recombination modulation factor is a genetic modification introduced by a site-specific nucleobase editor without a double strand DNA break.

40. The method of claim 36, wherein the modulated recombination is an increase in meiotic recombination frequency or meiotic cross-over events across whole genome or a portion of the genome.

41. The method of claim 36, wherein the modulated recombination is a decrease in meiotic recombination frequency or meiotic cross-over events across whole genome or a portion of the genome.

42. (canceled)
